(19) United States
(12) Patent Application Publication
(10) Pub. No.: US 2025/0092001 A1
(43) Pub. Date: Mar. 20, 2025

(54) NOVEL EXCITATORY AMINO ACID GLUTAMATE TRANSPORT MODULATORS AND METHODS USING THE SAME

(71) Applicant: Drexel University, Philadelphia, PA (US)

(72) Inventor: Sandhya Kortagere, Newtown, PA (US)

(21) Appl. No.: 18/832,382

(22) PCT Filed: Jan. 24, 2023
(86) PCT No.: PCT/US23/11385

§ 371 Date: Jul. 23, 2024

Related U.S. Application Data
(60) Provisional application No. 63/302,758, filed on Jan. 25, 2022.

(51) Int. Cl. C07D 295/12; A61K 31/495; A61P 25/28; C07D 295/16

(52) U.S. Cl. CPC C07D 295/12; A61K 31/495; A61P 25/28; C07D 295/16

(57) ABSTRACT

Described herein are compounds useful as glutamate transport modulators, such as GLT-1 modulators. Also described herein is a method of increasing glutamate clearance by a GLT-1 containing cell, the method including contacting the cell with a compound contemplated herein. Also described herein is a method of preventing, treating and/or ameliorating a neurological disorder in a subject in need thereof, the method including administering to the subject an effective amount of a compound contemplated herein.

### CROSS-REFERENCE TO RELATED APPLICATIONS

This application claims priority to U.S. Provisional Patent Application No. 63/302,758 entitled “NOVEL EXCITATORY AMINO ACID GLUTAMATE TRANSPORT MODULATORS AND METHODS USING THE SAME,” filed Jan. 25, 2022, the disclosure of which is incorporated herein by reference in its entirety.

### BACKGROUND

Glutamate is the predominant excitatory amino acid neurotransmitter in the mammalian central nervous system (CNS) and is essential for normal brain function, including cognition, memory, learning, developmental plasticity and long-term potentiation. The termination of glutamate neurotransmission is achieved by rapid uptake of the released glutamate by presynaptic and astrocytic sodium-dependent transporters. There are five subtypes of excitatory amino-acid transporters or glutamate transporters: EAAT1 or GLAST, EAAT2 or GLT-1, EAAT3 or EAAC1, EAAT4, and EAAT5. The major regulator of extracellular glutamate levels in the brain is EAAT2, which is expressed in astroglial cells and responsible for about 90% total glutamate uptake in the CNS and about 1% of total brain protein in the CNS.

Glutamate translocation is a multi-step process that uses energy derived from Na⁺ and K⁺ electrochemical gradients to move glutamate against its concentration gradient. The rate-limiting step in the cycle involves translocation of K⁺ and reorientation of the carrier to become accessible for a new substrate molecule in the synaptic cleft. Despite the identification of several crystal structures of a bacterial homolog of glutamate transporters Glt(ph), no rationally designed transporter activator has been identified so far.

Ischemic events in humans and in animals lead to an acute and sustained increase in extracellular glutamate concentrations, indicative of a lack of timely clearance by glutamate transporters. In fact, dysfunctional glutamate transporters are often the initiating event or part of the cascade leading to brain injury. Reductions in EAAT2 activity result in increased predisposition for seizures and susceptibility to damage due to ischemia. Excessive activation of glutamate receptors due to sustained elevation of extracellular glutamate levels results in Ca²⁺ influx and activation of a cascade of phospholipases, endonucleases and proteases such as calpain that can lead to apoptotic or necrotic cell death.

Glutamate excitotoxicity has been implicated in acute pathologies, such as traumatic brain injury, hyperexcitability, seizures and epilepsy, stroke, cerebral and retinal ischemia, and chronic pathologies, such as Parkinson's disease, amyotrophic lateral sclerosis (ALS), Alzheimer's disease, Huntington's disease, schizophrenia, neuropathic pain, alcohol and drug addictions, HIV-associated neurocognitive disorder, depression, learning and memory, impulsive and compulsive drug seeking behaviors associated with neurological and neurodegenerative disorders. Also, ischemic events in humans and animals lead to an acute and sustained increase in extracellular glutamate concentrations, which suggests a lack of proper clearance by glutamate transporters. In fact, dysfunctional glutamate transporters are often the initiating event or part of the cascade leading to brain injury.

Reductions in EAAT2 activity result in increased predisposition for seizures and susceptibility to damage due to ischemia. Knockout of the EAAT2 gene resulted in exacerbated damage compared to their wild-type counterparts following cerebral injury in mice and controlled cortical impact in rats. Additionally, a transgenic approach for EAAT2 overexpression with double transgenic mice created from crossing an ALS mouse model to a mouse model overexpressing EAAT2 resulted in animals that display delayed grip strength decline, motor neuron loss, and increased life expectancy.

Several classes of compounds that target stages in these processes, such as NMDA receptor antagonists and calcium influx inhibitors, alleviate cellular damage and neurologic deficits to some extent, but have limited clinical use due to substantial side effects. Transcriptional or translational upregulators of EAAT2, such as GPI-1046, ceftriaxone, harmine and pyridazine derivatives, are neuroprotective through selective augmentation of EAAT2 expression. Nevertheless, these compounds must be administered prophylactically to be neuroprotective, and thus they have low clinical relevance for acute conditions. Some compounds with neuroprotective properties, such as MS-153, riluzole, guanosine and nicergoline, acutely stimulate glutamate uptake by an indirect modulation of transporter activity, but are non-specific and cause numerous side effects.

There is a need in the art to identify molecules that regulate glutamate transmission and are useful for treatment, amelioration, and/or prevention of diseases or disorders that are caused, induced, or characterized by abnormal reduction in glutamate transporter activity or abnormal increase in extracellular CNS glutamate concentration in a subject. The present invention addresses this need.

### SUMMARY

In some aspects, the present invention is directed to the following non-limiting embodiments:

In some embodiments, the present invention is directed to a compound.

In some embodiments, the compound is a compound of Formula (I):

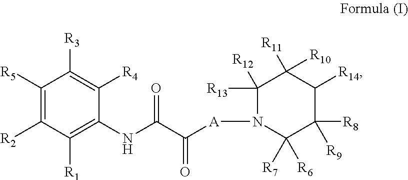

or a salt, solvate, enantiomer, diastereoisomer, isotopically labelled derivative, tautomer, or geometric isomer thereof, or mixtures thereof,

In some embodiments, A is a bond or

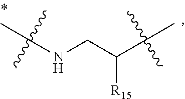

wherein * is the bond to the carbonyl group.

In some embodiments, R₁-R₁₃ are each independently selected from the group consisting of H, halogen, OH, C₁-C₆ alkyl, C₁-C₆ alkoxy, C₁-C₆ heteroalkyl, and C₃-C₁₀ cycloalkyl, wherein the alkyl, alkoxy, heteroalkyl, and cycloalkyl are each independently optionally substituted with at least one of C₁-C₆ alkyl, halogen, OH, and C₁-C₆ alkoxy.

In some embodiments, R₁₄ is H, C₁-C₆ alkyl, C₁-C₆ heteroalkyl, C₃-C₁₀ cycloalkyl, and phenyl, wherein the alkyl, alkoxy, heteroalkyl, and cycloalkyl are independently optionally substituted with at least one of C₁-C₆ alkyl, halogen, OH, and C₁-C₆ alkoxy.

In some embodiments, R₁₅ is phenyl optionally substituted with at least one of C₁-C₆ alkyl, halogen, OH, and C₁-C₆ alkoxy.

In some embodiments, the compound prevents, treats and/or ameliorates a neurological disorder.

In some embodiments, one or more of R₁-R₅ are C₁-C₆ alkoxy.

In some embodiments, R₁₄ is optionally substituted phenyl.

In some embodiments, the compound is at least one selected from the group consisting of N1-(4-methoxyphenyl)-N2-(2-(4-methylpiperazin-1-yl)-2-phenylethyl)oxalamide

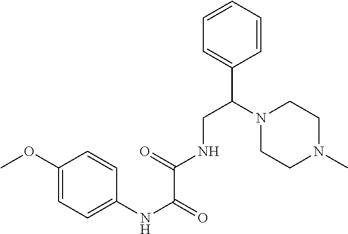

(GT467 or GTS467) and N-(4-methoxyphenyl)-2-oxo-2-(4-phenylpiperazin-1-yl)acetamide

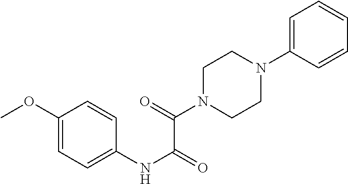 [SMILES: COc1ccc(NC(=O)C(=O)N2CCN(c3ccccc3)CC2)cc1]

(GT511), or a salt, solvate, enantiomer, diastereoisomer, isotopically labelled derivative, tautomer, or geometric isomer thereof, or mixtures thereof.

In some embodiments, the compound is (R)—N1-(4-methoxyphenyl)-N2-(2-(4-methylpiperazin-1-yl)-2-phenylethyl)oxalamide

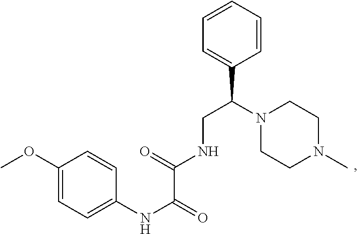

or (S)—N1-(4-methoxyphenyl)-N2-(2-(4-methylpiperazin-1-yl)-2-phenylethyl)oxalamide

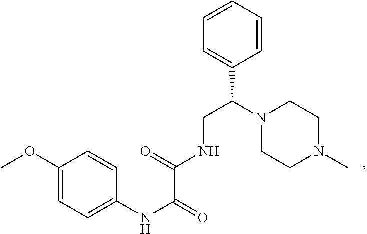

or a salt, solvate, isotopically labelled derivative, or tautomer thereof.

In some embodiments, the present invention is directed to a method of increasing activity of GLT-1 protein.

In some embodiments, the method comprising contacting the GLT-1 protein with the compound herein.

In some embodiments, the GLT-1 protein is an isolated protein or a protein expressed in a cell.

In some embodiments, the GLT-1 protein is on a surface of an astrocyte.

In some embodiments, the astrocyte is in a central nervous system (CNS) of a subject.

In some embodiments, the subject suffers from a condition associated with excessive extracellular glutamate concentration at a synapse of the CNS. In some embodiments, the method comprises administering to the subject an effective amount of the compound herein.

In some embodiments, the condition associated with excessive extracellular glutamate concentration at the synapse of the CNS comprises at least one neurological disorder selected from the group consisting of Parkinson's disease, Alzheimer's disease, Huntington's disease, amyotrophic lateral sclerosis (ALS), stroke, epilepsy, schizophrenia, compulsive and impulsive drug and alcohol seeking behaviors, and learning and memory impairment associated with neurological and neuropsychiatric disorders.

In some embodiments, the present invention is directed to a method of preventing, treating and/or ameliorating neurological disorder in a subject in need thereof.

In some embodiments, the method comprises administering to the subject an effective amount of the compound herein.

In some embodiments, the neurological disorder comprises at least one selected from the group consisting of Parkinson's disease, Alzheimer's disease, Huntington's disease, amyotrophic lateral sclerosis (ALS), stroke, epilepsy, schizophrenia, compulsive and impulsive drug and alcohol seeking behaviors, and/or learning and memory impairment associated with neurological and neuropsychiatric disorders.

In some embodiments, the compound is administered to the subject as a pharmaceutical composition.

In some embodiments, the pharmaceutical composition further comprises a pharmaceutically acceptable carrier.

In some embodiments, the method further comprises administering to the subject an additional therapeutic agent.

In some embodiments, the compound and the additional therapeutic agent are co-administered to the subject.

In some embodiments, the compound and the additional therapeutic agent are co-formulated.

In some embodiments, the subject is a human.

### BRIEF DESCRIPTION OF THE DRAWINGS

The following detailed description of exemplary embodiments will be better understood when read in conjunction with the appended drawings. For the purpose of illustrating, non-limiting embodiments are shown in the drawings. It should be understood, however, that the instant specification is not limited to the precise arrangements and instrumentalities of the embodiments shown in the drawings.

FIG. 1 is a non-limiting proposed mechanism of action of the GLT-1 modulators in accordance with some embodiments. Specifically, in normal conditions, glutamate (Glu) is released from neuronal terminals and is taken up by GLT-1 (also known as EAAT2) on astrocytes. Glu is converted to glutamine (Gln) in the astrocytes and transported out of the astrocyte. Gln is then packaged by VGlut2 in the neuron (not shown). In disease conditions such as Parkinson's disease, loss of dopamine neurons (DA neurons) leads to production and release of excess Glu and down regulation of GLT-1 (EAAT2). The GLT-1 modulators described herein activate GLT-1 (EAAT2) and increase the uptake of Glu by astrocytes.

FIG. 2 depicts the results of a glutamate uptake assay testing nine (9) novel compounds as well as the parent compound, in accordance with some embodiments.

FIG. 3 shows the chemical structures of two exemplary GLT-1 modulators in accordance with some embodiments. Also shown are the EC₅₀ values of the exemplary GLT-1 modulators.

FIGS. 4A and 4B depict the pharmacokinetic profile of two exemplary GLT-1 modulators in plasma of rats when administer intraperitoneally (IP), intravenously (IV) or orally (PO), in accordance with some embodiments. FIG. 4A depicts the pharmacokinetic profile of GT467. FIG. 4B depicts the pharmacokinetic profile of GT511.

FIGS. 5A and 5B depict the pharmacokinetic profile of two exemplary GLT-1 modulators in brain of rats when administer intraperitoneally (IP), intravenously (IV) or orally (PO), in accordance with some embodiments. FIG. 5A depicts the pharmacokinetic profile of GT467. FIG. 5B depicts the pharmacokinetic profile of GT511.

FIGS. 6A-6C demonstrate that the exemplary GLT-1 modulators improve locomotive activities in a Drosophila of a Huntington's disease model, in accordance with some embodiments. FIG. 6A illustrate certain aspects of a locomotion assay used to assay the locomotive activities. As shown in FIG. 6A, Drosophila larvae were place on the central line of the dish, and numbers of lines crossed by the larvae in 30 seconds were recorded. FIGS. 6B and 6C depict the results of the locomotion assay results of wild-type Drosophila (“WT”), as well as Drosophila of a Huntington's disease model treated with vehicle (“Veh”) or with various concentrations of two exemplary GLT-1 modulators. FIG. 6B depicts the results of GT467 treatment. FIG. 6C depicts the results of GT511 treatment.

FIGS. 7A-7D demonstrate that the exemplary GLT-1 modulators improve learning and memory in the Drosophila of the Huntington's disease model, in accordance with some embodiments. FIG. 7A illustrates certain aspects of a larval olfactory associative learning used to test learning and memory. In FIG. 7A, larvae were trained with either a reinforcer solution containing sucrose (“LIN/SUC”) or a control of distilled water (“LIN/DW”). The trained larvae were then transferred to a dish having test odorant placed on one side and a control on the opposite side. Larvae that made the association with the odorant and crossed to the odorant side in <1 min were classified as high performers and those that crossed to the odorant side between 1-3 min as low performers and those larvae that did not make the association were called non-performers. The ΔRI (difference between LIN/SUC and LIN/DW performance scores) were then calculated for the high performers (FIG. 7B), low performers (FIG. 7C) and non-performers (FIG. 7D).

FIGS. 8A-8B demonstrate that exemplary GLT-1 modulators improve longevity in the Drosophila of the Huntington's disease model, in accordance with some embodiments. The longevity assay was performed according to Romero et al., Neuron, 57(1), 27-40 and Linford et al., JoVE (Journal of Visualized Experiments), (71). Briefly, 100 adult flies from wild type and HTT phenotype (male and female) were collected in each designated tube and fed with food containing the specific concentration of drug. Every 2-3 days the flies were moved into new vials with fresh food and drug. Dead flies were removed and the number of flies alive during each change is noted. The tunes were maintained until the last available fly and the data was plotted as a survival curve. FIG. 8A illustrates certain aspects of a longevity assay. FIG. 8B depicts the results of the longevity assay.

FIGS. 9A-9B demonstrate that an exemplary GLT-1 modulator rescued locomotor deficits and improved performance in memory task in mice of a Huntington disease model (R6/2 mice), in accordance with some embodiments. FIG. 9A depicts the results of a locomotion test. In the locomotion test of FIG. 9A, wildtype and R6/2 mice treated with vehicle or different doses of GTS467 (2, 4, and 10 mg/kg) or ceftriaxone (200 mg/kg) were subjected to the open field test to evaluate their locomotor abilities in a 44×44 cm and 43×43 cm square open field arena. Animals were placed individually in the open field arena and behavior was recorded for a period of 5 minutes under dim light and low noise conditions. Animal behavior was recorded using an overhead camera and the distance moved was recorded and quantified using the Noldus ethovision XT-14 software. FIG. 9B depicts the results of a memory task test. Referring to FIG. 9B, to test for memory impairment a novel object recognition assay was performed. A 44×44 cm square shaped open field arena was used to perform this test at week 12 and week 15. The behavioral evaluation was performed in 2 sets, each set having equal number of animals. The animals were subjected to habituation a day prior to the commencement of test for 5 min followed by familiarization with similar objects for 10 min and tested with different objects for 10 min on the following day. The behavior was recorded under dim light and low noise conditions using an overhead camera. Cumulative duration with both the objects, total object exploration (sec), distance travelled (cm) was quantified using the Noldus ethovision XT 14 software.

FIG. 10 is a diagram illustrating that GTS467 activates EAAT2 and regulates glutamate neurotransmission to reduce excitotoxicity, in accordance with some embodiments.

FIG. 11 is a diagram illustrating certain aspects of the experimental design of the Example 2, in accordance with some embodiments.

FIG. 12 lists the training schedule for 5CSRTT (5-Choice Serial Reaction Time Task) assay with criteria for success, in accordance with some embodiments.

FIG. 13: Schematic representation of the 5CSRTT assay, in accordance with some embodiments. Rats were trained to receive food reward for a correct nose poke response to a signal light (correct trial shown in arrow connecting 1 and 3). Incorrect trials (the arrows connecting 2/3 to 1) or omissions (the arrow connecting 4 to other boxes) results in time out indicated by darkness with no Cue lights. Trial resets with variable intertrial intervals. Nose pokes made during inter trial interval (ITI) are recorded as premature responses (PMR) that codes for impulsive behavior

FIGS. 14A-14C: Results of 5CSRTT, in accordance with some embodiments. PD rats treated with GTS467 (GTS) significantly reduced the premature responses (PMR) compared to Saline (FIG. 14AA). Further GTS467 made significantly higher correct choices (CR) (FIG. 14B) and lower Omission (OM) (FIG. 14C) compared to saline *P<0.05.

FIGS. 15A-15B: Frequency and Time spent in the open and close arm of EPM by PD rats treated with either saline or GTS467, in accordance with some embodiments. The results show all animal groups had significant differences in the frequency of entering the open arm (FIG. 15A) but no significant changes in time spent in the open arm (FIG. 15B) suggesting that the drugs did not induce any anxiety. *, #P<0.05, for saline and GTS467, respectively.

FIG. 16: Representative immunoblot of EAAT2, NR2A, NR2B, NR1 along with the loading control β-actin in cortex (CRTX) and striatum (STR) from PD rats post behavior studies, in accordance with some embodiments.

FIG. 17: Quantification of the immunoblot in the upper panel showing significant (*P<0.05, **P<0.01) increase in the expression upon GTS467 treatment except NR1 which reduced in CRTX, in accordance with some embodiments. Left panels—CRTX, Right panels—STR.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Current, small molecule therapies available to treat glutamate disorders act mostly on neuronal targets, such as metabotropic glutamate receptors (mGluRs), N-methyl-D-aspartate receptors (NMDA receptors or NMDARs) and α-amino-3-hydroxy-5-methyl-4-isoxazolepropionic acid receptors (AMPA receptors or AMPARs). mGluRs, NMDARs, and AMPARs; however, are present on neurons and directly involved in the synaptic transmission. As such, modulating the activity of these neuronal targets often lead to unwanted, sometimes severe side effects that limit the utility of these small molecule therapies for treating a wide range of glutamate disorders.

GLT-1 (glutamate transporter 1, also known as excitatory amino acid transporter 2 or EAAT2) is a transporter that clears the excitatory neurotransmitter glutamate from the extracellular space at synapses. GLT-1 is responsible for nearly 85% of all glutamate transmission in the brain. Unlike the conventional neuronal targets mGluRs, NMDARs, and AMPARs, GLT-1 is predominantly distributed on astrocytes (although a small portion of protein is distributed on neurons in a region-specific manner).

The study described herein (“the present study”) identifies novel modulators of GLT-1. The small molecule modulators identified in the present study upregulate GLT-1 by increasing the activity of the glutamate transporter. Activated GLT-1 on a cell increases the rate of glutamate transportation, thereby transporting extracellular glutamate into the cell at an increased rate. Since GLT-1 is mostly present on astrocytes, the upregulation of GLT-1 by the GLT-1 modulators described herein allow the astrocytes to scavenge out excess glutamate and reduce the concentration of glutamate near synapses.

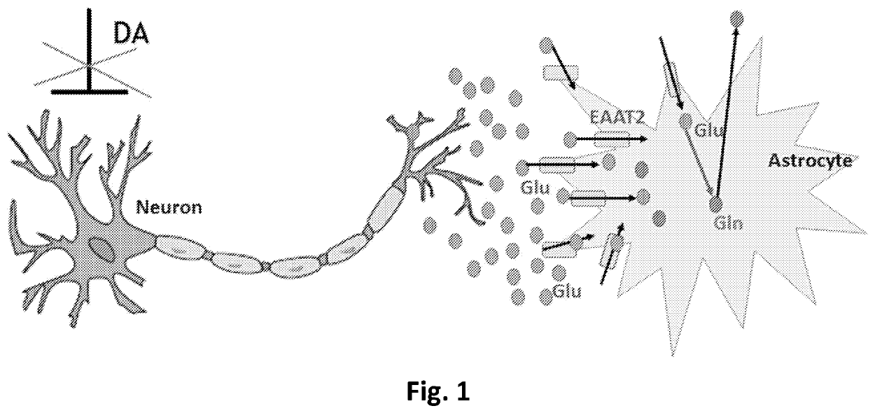

Referring to FIG. 1, the activation of GLT-1 (also called EAAT2) on the astrocytes allows the excess glutamate, which can cause neurological conditions, to enter the astrocyte via the activated GLT-1. The glutamate is then converted to glutamine in the astrocytes by enzymes such as glutamine synthetase (GS). In neurodegenerative diseases, this can lead to improvements in symptoms such as motor deficits and treatment of chorea and cognitive impairment. Furthermore, due to the fact that GLT-1 expression in neurons are limited, the GLT-1 activators discovered by the present study can reduce excitotoxicity without directly affecting the synaptic transmission at the pre- and post-synaptic sites. Because the GLT-1 activators of the invention have the ability to scavenge out excess glutamate without interfering with the neuronal glutamate receptors, these GLT-1 activators provide significant advantages in efficacy and reduction of side effects.

Accordingly, in some aspects, the present invention is directed to a compound, wherein the compound is a compound represented by Formula (I), or a salt, solvate, enantiomer, diastereoisomer, isotopically labelled derivative, tautomer, or geometric isomer thereof, or mixtures thereof:

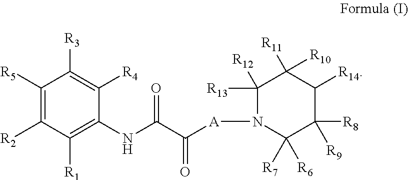

In some embodiments, the compound of the present invention is useful as a glutamate transport modulator, such as a GLT-1 modulator.

In some aspects, the present invention is directed to a method of increasing GLT-1 activity. The method includes contacting the GLT-1 with a compound of Formula (I), or a salt, solvate, enantiomer, diastereoisomer, isotopically labelled derivative, tautomer, or geometric isomer thereof, or mixtures thereof.

In some aspects, the present invention is directed to a method of increasing glutamate clearance by a GLT-1 containing cell. The method includes contacting the GLT-1 containing cell with a compound of Formula (I), or a salt, solvate, enantiomer, diastereoisomer, isotopically labelled derivative, tautomer, or geometric isomer thereof, or mixtures thereof.

In some aspects, the present invention is directed to a method of preventing, treating and/or ameliorating a neurological disorder in a subject in need thereof. The method includes administering to the subject an effective amount of a compound of Formula (I), or a salt, solvate, enantiomer, diastereoisomer, isotopically labelled derivative, tautomer, or geometric isomer thereof, or mixtures thereof. In some embodiments, the neurological disorder includes Parkinson's disease, Alzheimer's disease, Huntington's disease, amyotrophic lateral sclerosis (ALS), stroke, epilepsy, and/or schizophrenia, compulsive and impulsive drug and alcohol seeking behaviors, learning and memory impairment associated with neurological and neuropsychiatric disorders.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

“About” as used herein when referring to a measurable value such as an amount, a temporal duration, and the like, is meant to encompass variations of ±20% or ±10%, in certain embodiments ±5%, in certain embodiments ±1%, in certain embodiments ±0.1% from the specified value, as such variations are appropriate to perform the disclosed methods.

As used herein, the term “activator” or “stimulator” or “upregulator” as applied to a glutamate transporter refers to a compound that interacts with a glutamate transporter, such as a glutamate transporter in a cell or a subject and increases, upregulates, enhances, and/or restores the activity of the transporter. In certain embodiments, a glutamate transporter activator regulates extracellular glutamate concentrations in a cell or a subject in need thereof. The normal activity level of a glutamate transporter may be determined by examining a cell or subject with a normally functioning glutamate transporter. An abnormal reduction in glutamate transporter activity or an abnormal increase in extracellular CNS glutamate concentration may cause a disease or disorder in the subject. In one aspect, dysfunctional glutamate transporters are often the initiating event or part of the cascade leading to brain injury, and reductions in glutamate transport activity result in increased predisposition for seizures and susceptibility to damage due to ischemia.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Illustrative examples are C₁-C₃ alkoxy, particularly ethoxy and methoxy.

As used herein, the term “alkyl” by itself or as part of another substituent means, unless otherwise stated, a straight or branched chain hydrocarbon having the number of carbon atoms designated (i.e., C₁₋₆ means one to six carbon atoms) and includes straight, branched chain, or cyclic substituent groups. Examples include methyl, ethyl, propyl, isopropyl, butyl, isobutyl, tert-butyl, pentyl, neopentyl, hexyl, and cyclopropylmethyl. Examples include C₁-C₆ alkyl, particularly ethyl, methyl, isopropyl, isobutyl, n-pentyl, n-hexyl and cyclopropylmethyl. C₀ alkyl corresponds to a bond.

As used herein, the term “alkenyl,” employed alone or in combination with other terms, means, unless otherwise stated, a stable monounsaturated or diunsaturated straight chain or branched chain hydrocarbon group having the stated number of carbon atoms. Examples include vinyl, propenyl (or allyl), crotyl, isopentenyl, butadienyl, 1,3-pentadienyl, 1,4-pentadienyl, and the higher homologs and isomers. A functional group representing an alkene is exemplified by —CH₂—CH═CH₂.

As used herein, the term “alkynyl,” employed alone or in combination with other terms, means, unless otherwise stated, refers to a straight-chain or branched hydrocarbon residue having a carbon-carbon triple bond and have the number of carbon atoms designated (i.e., C₁-C₁₀ means one to ten carbon atoms) and includes straight, branched chain, or cyclic substituent groups.

As used herein, the term “cycloalkyl” by itself or as part of another substituent refers to, unless otherwise stated, a cyclic chain hydrocarbon having the number of carbon atoms designated (i.e., C₃-C₆ refers to a cyclic group comprising a ring group consisting of three to six carbon atoms) and includes straight, branched chain or cyclic substituent groups. Examples of (C₃-C₆)cycloalkyl groups are cyclopropyl, cyclobutyl, cyclopentyl and cyclohexyl. Cycloalkyl rings can be optionally substituted. Non-limiting examples of cycloalkyl groups include: cyclopropyl, 2-methyl-cyclopropyl, cyclopropenyl, cyclobutyl, 2,3-dihydroxycyclobutyl, cyclobutenyl, cyclopentyl, cyclopentenyl, cyclopentadienyl, cyclohexyl, cyclohexenyl, cycloheptyl, cyclooctanyl, decalinyl, 2,5-dimethylcyclopentyl, 3,5-dichlorocyclohexyl, 4-hydroxycyclohexyl, 3,3,5-trimethylcyclohex-1-yl, octahydropentalenyl, octahydro-1H-indenyl, 3a,4,5,6,7,7a-hexahydro-3H-inden-4-yl, decahydroazulenyl; bicyclo[6.2.0]decanyl, decahydronaphthalenyl, and dodecahydro-1H-fluorenyl. The term “cycloalkyl” also includes bicyclic hydrocarbon rings, non-limiting examples of which include, bicyclo[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

As used herein, the term “halo” or “halogen” alone or as part of another substituent refers to, unless otherwise stated, a fluorine, chlorine, bromine, or iodine atom.

As used herein, the term “substituted” refers to that an atom or group of atoms has replaced hydrogen as the substituent attached to another group.

As used herein, the term “substituted alkyl,” “substituted cycloalkyl,” “substituted alkenyl,” or “substituted alkynyl” refers to alkyl, cycloalkyl, alkenyl, or alkynyl, as defined elsewhere herein, substituted by one, two or three substituents independently selected from the group consisting of halogen, —OH, alkoxy, tetrahydro-2-H-pyranyl, —NH₂, —NH(C₁-C₆ alkyl), —N(C₁-C₆ alkyl)₂, 1-methyl-imidazol-2-yl, pyridin-2-yl, pyridin-3-yl, pyridin-4-yl, —C(═O)OH, —C(═O)O(C₁-C₆)alkyl, trifluoromethyl, —C≡N, —C(═O)NH₂, —C(═O)NH(C₁-C₆)alkyl, —C(═O)N((C₁-C₆)alkyl)₂, —SO₂NH₂, —SO₂NH(C₁-C₆ alkyl), —SO₂N(C₁-C₆ alkyl)₂, —C(═NH)NH₂, and —NO₂, in some embodiments containing one or two substituents independently selected from halogen, —OH, alkoxy, —NH₂, trifluoromethyl, —N(CH₃)₂, and —C(═O)OH, in some embodiments independently selected from halogen, alkoxy and —OH. Examples of substituted alkyls include, but are not limited to, 2,2-difluoropropyl, 2-carboxycyclopentyl and 3-chloropropyl.

Whenever a term or either of their prefix roots appear in a name of a substituent the name is to be interpreted as including those limitations provided herein. For example, whenever the term “alkyl” or “aryl” or either of their prefix roots appear in a name of a substituent (e.g., arylalkyl, alkylamino) the name is to be interpreted as including those limitations given elsewhere herein for “alkyl” and “aryl” respectively.

In some embodiments, substituents of compounds are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C_{6,} C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

As used herein, the term “aromatic” refers to a carbocycle or heterocycle with one or more polyunsaturated rings and having aromatic character, i.e., having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl, and naphthyl. Illustrative examples are phenyl and naphthyl, or phenyl.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a disease or disorder in a patient.

As used herein, the terms “effective amount” and “pharmaceutically effective amount” and “therapeutically effective amount” refer to a nontoxic but sufficient amount of an agent or compound to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

As used herein, the term “halo” or “halogen” alone or as part of another substituent means, unless otherwise stated, a fluorine, chlorine, bromine, or iodine atom, for example, fluorine, chlorine, or bromine, for example, fluorine or chlorine.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, such as between the rest of the heteroalkyl group and the fragment to which it is attached, as well as attached to the most distal carbon atom in the heteroalkyl group. Examples include —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂NH—OCH₃, or —CH₂CH₂—SS—CH₃

As used herein, the term “heterocycle” or “heterocyclyl” or “heterocyclic” by itself or as part of another substituent means, unless otherwise stated, an unsubstituted or substituted, stable, mono- or multi-cyclic heterocyclic ring system that consists of carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In one embodiment, the heterocycle is a heteroaryl.

As used herein, the term “heteroaryl” or “heteroaromatic” refers to a heterocycle having aromatic character. A polycyclic heteroaryl may include one or more rings that are partially saturated. Examples include tetrahydroquinoline and 2,3-dihydrobenzofuryl.

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

Examples of heteroaryl groups include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benztriazolyl, thioxanthinyl, carbazolyl, carbolinyl, acridinyl, pyrrolizidinyl, and quinolizidinyl.

The listing of heterocyclyl and heteroaryl moieties provided herein is intended to be representative and not limiting.

As used herein, the term “substituted” means that an atom or group of atoms has replaced hydrogen as the substituent attached to another group.

For cycloalkyl, aryl, heteroaryl, and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In certain embodiments, the substituents vary in number between one and four. In other embodiments, the substituents vary in number between one and three. In yet other embodiments, the substituents vary in number between one and two.

In some embodiments, the substituents are each independently selected from the group consisting of —C₁-C₆ alkyl, —OH, —C₁-C₆ alkoxy, halogen, —NH₂, —N(CH₃)₂, —C(═O)OH, trifluoromethyl, —C≡N, —C(═O)O(C₁-C₄)alkyl, —C(═O)NH₂, —NHC(═O)(aryl or alkyl), —SO₂NH₂, —C(═NH)NH₂, —NO₂, or any combinations thereof. In some embodiments, the substituents are independently selected from the group consisting of C₁-C₆ alkyl, C₁-C₆ alkoxy, halo, acetamido, and nitro. As used herein, where a substituent is an alkyl or alkoxy group, the carbon chain may be branched, straight or cyclic, with straight being an illustrative example.

As used herein, the term “GT467” or “GTS467” refers to N1-(4-methoxyphenyl)-N2-(2-(4-methylpiperazin-1-yl)-2-phenylethyl)oxalamide

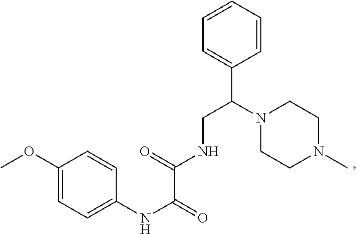

or an enantiomer, salt, or solvate thereof.

As used herein, the term “GT511” refers to N-(4-methoxyphenyl)-2-oxo-2-(4-phenylpiperazin-1-yl)acetamide

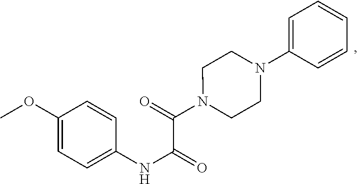 [SMILES: COc1ccc(NC(=O)C(=O)N2CCN(c3ccccc3)CC2)cc1]

or an enantiomer, salt, or solvate thereof.

As used herein, the terms “patient” and “subject” and “individual” refer interchangeably to a human or a non-human mammal. Non-human mammals include, for example, livestock and pets, such as ovine, bovine, porcine, canine, feline and murine mammals. For example, the patient or subject is human.

As used herein, the term “pharmaceutical composition” refers to a mixture of at least one compound useful in the methods of the invention with a pharmaceutically acceptable carrier. The pharmaceutical composition facilitates administration of the compound to a patient. Multiple techniques of administering a compound exist in the art including, but not limited to, intravenous, intramuscular, oral, aerosol, parenteral, ophthalmic, pulmonary and topical administration.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful in the methods of the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful in the methods of the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful in the methods of the invention, and are physiologically acceptable to the patient. Supplementary active compounds may also be incorporated into the compositions. The “pharmaceutically acceptable carrier” may further include a pharmaceutically acceptable salt of the compound useful in the methods of the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “salts” embraces addition salts of free acids or bases that are useful within the methods of the invention. In certain embodiments, the salts are pharmaceutically acceptable salts. The language “pharmaceutically acceptable salt” refers to a salt of the administered compounds prepared from pharmaceutically acceptable non-toxic acids or bases, including inorganic acids or bases, organic acids or bases, and solvates, hydrates, or clathrates thereof. Pharmaceutically unacceptable salts may nonetheless possess properties such as high crystallinity, which have utility in the practice of the present invention, such as for example utility in process of synthesis, purification or formulation of compounds useful within the methods of the invention.

Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxy benzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluene sulfonic, sulfanilic, cyclohexylaminosulfonic, stearic, alginic, β-hydroxybutyric, salicylic, galactaric and galacturonic acid.

Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “treatment” or “treating,” is defined as the application or administration of a therapeutic compound, i.e., a compound of the invention (alone or in combination with another therapeutic agent), to a patient, or application or administration of a therapeutic compound to an isolated tissue or cell line from a patient (e.g., for diagnosis or ex vivo applications), who has a disease or disorder and/or a symptom of a disease or disorder, with the purpose to cure, heal, alleviate, relieve, alter, remedy, ameliorate, improve, or affect a disease or disorder and/or the symptoms of a disease or disorder. Such treatments may be specifically tailored or modified, based on knowledge obtained from the field of pharmacogenomics.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compounds

In aspects, the present invention is directed to a compound. In some embodiments, the compound is a glutamate transport modulator, such as a GLT-1 modulator that is able to modulate the activity of GLT-1 protein. In some embodiments, the compound is a glutamate transport activator, such as a GLT-1 activator that is able to increase or upregulate the activity of GLT-1 protein

The compound can be synthesized using techniques well-known in the art of organic synthesis, such as those detailed below in the “Synthesis of Compound” section.

In some embodiments, the compound is a compound of Formula (I):

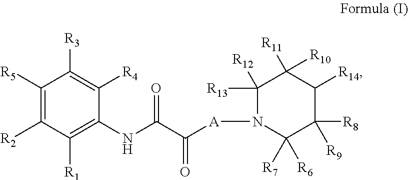

or a salt, solvate, enantiomer, diastereoisomer, isotopically labelled derivative, tautomer, or geometric isomer thereof, or mixtures thereof,
wherein in Formula (I):
A is a bond or

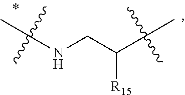

wherein * is the bond to the carbonyl group;
R₁-R₁₃ are each independently selected from the group consisting of H, halogen, OH, C₁-C₆ alkyl, C₁-C₆ alkoxy, C₁-C₆ heteroalkyl, and C₃-C₁₀ cycloalkyl, wherein the alkyl, alkoxy, heteroalkyl, and cycloalkyl are each independently optionally substituted, such as independently substituted with at least one of C₁-C₆ alkyl, halogen, OH, and C₁-C₆ alkoxy;
R₁₄ is H, C₁-C₆ alkyl, C₁-C₆ heteroalkyl, C₃-C₁₀ cycloalkyl, and phenyl, wherein the alkyl, alkoxy, heteroalkyl, and cycloalkyl are independently optionally substituted with at least one of C₁-C₆ alkyl, halogen, OH, and C₁-C₆ alkoxy;
R₁₅ is phenyl optionally substituted with at least one of C₁-C₆ alkyl, halogen, OH, and C₁-C₆ alkoxy.

In some embodiments, one or more of R₁-R₅ are C₁-C₆ alkoxy. In some embodiments, one or more of R₁-R₅ is C₁-C₆ alkoxy, and the rest of R₁-R₅ are H.

In some embodiments, one or more of R₆-R₁₃ are H. In some embodiments, all of R₆-R₁₃ are H.

In some embodiments, R₁₄ is optionally substituted phenyl.

In some embodiments, the compound is N1-(4-methoxyphenyl)-N2-(2-(4-methylpiperazin-1-yl)-2-phenylethyl)oxalamide

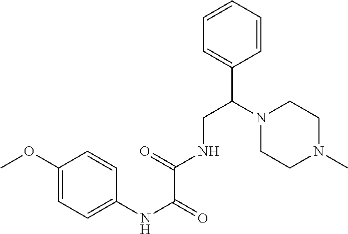

(GT467) or N-(4-methoxyphenyl)-2-oxo-2-(4-phenylpiperazin-1-yl)acetamide

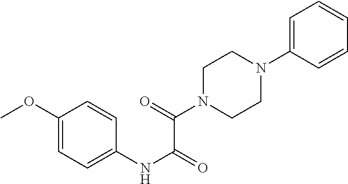 [SMILES: COc1ccc(NC(=O)C(=O)N2CCN(c3ccccc3)CC2)cc1]

(GT511), or a salt, solvate, enantiomer, diastereoisomer, isotopically labelled derivative, tautomer, or geometric isomer thereof, or mixtures thereof.

In some embodiments, the compound is (R)—N1-(4-methoxyphenyl)-N2-(2-(4-methylpiperazin-1-yl)-2-phenylethyl)oxalamide

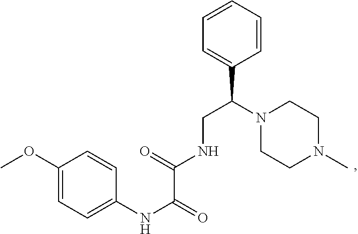

or (S)—N1-(4-methoxyphenyl)-N2-(2-(4-methylpiperazin-1-yl)-2-phenylethyl)oxalamide

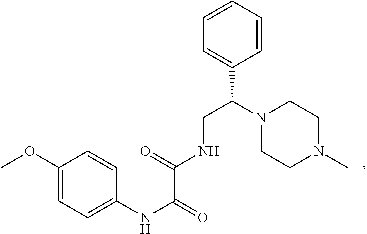

The compounds of the invention may possess one or more stereocenters, and each stereocenter may exist independently in either the (R)- or (S)-configuration. In certain embodiments, compounds described herein are present in optically active or racemic forms. The compounds described herein encompass racemic, optically active, regioisomeric and stereoisomeric forms, or combinations thereof that possess the therapeutically useful properties described herein. Preparation of optically active forms is achieved in any suitable manner, including, by way of non-limiting example, by resolution of the racemic form with recrystallization techniques, synthesis from optically active starting materials, chiral synthesis, or chromatographic separation using a chiral stationary phase. A compound illustrated herein by the racemic formula further represents either of the two enantiomers or any mixtures thereof, or in the case where two or more chiral centers are present, all diastereomers or any mixtures thereof.

In certain embodiments, the compounds of the invention exist as tautomers. All tautomers are included within the scope of the compounds recited herein.

Compounds described herein also include isotopically labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, substitution with heavier isotopes such as deuterium affords greater chemical stability. Isotopically labeled compounds are prepared by any suitable method or by processes using an appropriate isotopically labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

In all of the embodiments provided herein, examples of suitable optional substituents are not intended to limit the scope of the claimed invention. The compounds of the invention may contain any of the substituents, or combinations of substituents, provided herein.

### Salts

The compounds described herein may form salts with acids or bases, and such salts are included in the present invention. The term “salts” embraces addition salts of free acids or bases that are useful within the methods of the invention. The term “pharmaceutically acceptable salt” refers to salts that possess toxicity profiles within a range that affords utility in pharmaceutical applications. In certain embodiments, the salts are pharmaceutically acceptable salts. Pharmaceutically unacceptable salts may nonetheless possess properties such as high crystallinity, which have utility in the practice of the present invention, such as for example utility in process of synthesis, purification or formulation of compounds useful within the methods of the invention.

Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (or pamoic), methanesulfonic, ethanesulfonic, benzenesulfonic, pantothenic, sulfanilic, 2-hydroxyethanesulfonic, trifluoromethanesulfonic, p-toluenesulfonic, cyclohexylaminosulfonic, stearic, alginic, β-hydroxybutyric, salicylic, galactaric, galacturonic acid, glycerophosphonic acids and saccharin (e.g., saccharinate, saccharate). Salts may be comprised of a fraction of one, one or more than one molar equivalent of acid or base with respect to any compound of the invention.

Suitable pharmaceutically acceptable base addition salts of compounds of the invention include, for example, ammonium salts and metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (or N-methylglucamine) and procaine. All of these salts may be prepared from the corresponding compound by reacting, for example, the appropriate acid or base with the compound.

### Synthesis of Compound

The instant specification further provides methods of preparing the compound of the instant specification. Compounds of the instant specification can be prepared in accordance with the procedures outlined herein, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field.

It is appreciated that where typical or preferred process conditions (i.e., reaction temperatures, times, mole ratios of reactants, solvents, pressures, and so forth) are given, other process conditions can also be used unless otherwise stated. Optimum reaction conditions can vary with the particular reactants or solvent used, but such conditions can be determined by one skilled in the art by routine optimization procedures. Those skilled in the art of organic synthesis will recognize that the nature and order of the synthetic steps presented can be varied for the purpose of optimizing the formation of the compounds described herein.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatography such as high-performance liquid chromatograpy (HPLC), gas chromatography (GC), gel-permeation chromatography (GPC), or thin layer chromatography (TLC).

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000, 2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In some embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In some embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In some embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

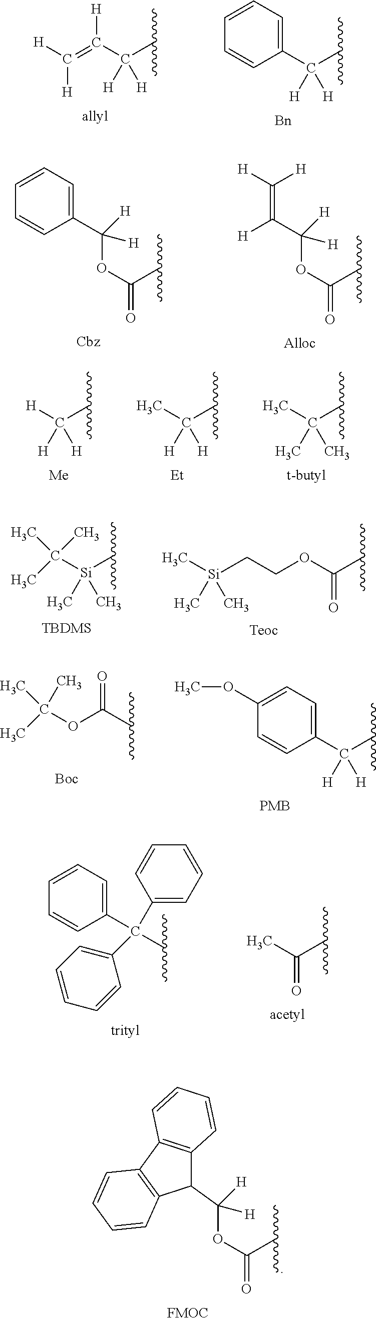

Other protecting groups, plus a detailed description of techniques applicable to the creation of protecting groups and their removal are described in Greene & Wuts, Protective Groups in Organic Synthesis, 3^{rd} Ed., John Wiley & Sons, New York, NY, 1999, and Kocienski, Protective Groups, Thieme Verlag, New York, NY, 1994, which are incorporated herein by reference for such disclosure.

### Composition

In some aspects, the present invention is directed to a pharmaceutical composition including a compound contemplated herein.

In some embodiments, the composition is a composition for increasing GLT-1 Activity. In some embodiments, the composition is useful for methods contemplated herein.

In some embodiments, the composition is a composition for increasing glutamate clearance by GLT-1 containing cell. In some embodiments, the composition is useful for methods contemplated herein.

In some embodiments, the composition is composition for preventing, treating and/or ameliorating a neurological disorder. In some embodiments, the composition is useful for methods contemplated herein.

### Method of Increasing GLT-1 Activity

In some aspects, the present invention is directed to a method of increasing GLT-1 activity. In some embodiments, the method includes contacting GLT-1 with a compound contemplated herein.

In some embodiments, the GLT-1 is an isolated protein. In some embodiments, the GLT-1 is on the surface of a cell. In some embodiments, the GLT-1 is introduced into the cell exogenously, such as expressed in cell using an expression vector, such as a plasmid or a viral vector. In some embodiments, the GLT-1 is expressed endogenously by the cell. In some embodiments, the GLT-1 is on the surface of an astrocyte.

In some embodiments, the GLT-1 is on the surface of an astrocyte, wherein the astrocyte is in the central nervous system (CNS) of a subject. In some embodiments, the subject is a mammal, such as a human. In some embodiments, the subject is suffering from a condition that would benefit from a reduced level of extracellular glutamate at a synapse of the subject, and the method includes administering to the subject an effective amount of the compound. In some embodiments, the condition that would benefit from reduced level of extracellular glutamate at the synapse is a neurological disorder, such as Parkinson's disease, Alzheimer's disease, Huntington's disease, amyotrophic lateral sclerosis (ALS), stroke, epilepsy, schizophrenia, compulsive and impulsive drug and alcohol seeking behaviors, and/or learning and memory impairment associated with neurological and neuropsychiatric disorders. In some embodiments, the compound is administered as part of a pharmaceutical composition.

### Method of Increasing Glutamate Clearance by GLT-1 Containing Cell

In some aspects, the present invention is directed to a method of increasing glutamate clearance by a cell, wherein the cell comprises GLT-1. In some embodiments, the method includes contacting the cell with a compound contemplated herein.

In some embodiments, the GLT-1 is introduced into the cell exogenously, such as expressed in cell using an expression vector (e.g., a plasmid or a viral vector). In some embodiments, the GLT-1 is expressed endogenously by the cell. In some embodiments, the GLT-1 is on the surface of an astrocyte.

In some embodiments, the GLT-1 is on the surface of an astrocyte, wherein the astrocyte is in the central nervous system (CNS) of a subject. In some embodiments, the subject is a mammal, such as a human. In some embodiments, the subject is suffering from a condition that would benefit from an increased glutamate clearance, such as increased glutamate clearance by the astrocyte, and the method includes administering to the subject an effective amount of the compound. In some embodiments, the condition that would benefit from the increased glutamate clearance is a neurological disorder, such as Parkinson's disease, Alzheimer's disease, Huntington's disease, amyotrophic lateral sclerosis (ALS), stroke, epilepsy, schizophrenia, compulsive and impulsive drug and alcohol seeking behaviors, and/or learning and memory impairment associated with neurological and neuropsychiatric disorders. In some embodiments, the compound is part of a pharmaceutical composition contemplated herein.

### Method of Preventing, Treating and/or Ameliorating Neurological Disorder

In some aspects, the present invention is directed to a method of preventing, treating and/or ameliorating neurological disorder in a subject in need thereof. In some embodiments, the method includes administering to the subject an effective amount of a compound contemplated herein.

In some embodiments, the neurological disorder includes Parkinson's disease, Alzheimer's disease, Huntington's disease, amyotrophic lateral sclerosis (ALS), stroke, epilepsy, schizophrenia, compulsive and impulsive drug and alcohol seeking behaviors, and/or learning and memory impairment associated with neurological and neuropsychiatric disorders.

In some embodiments, the neurological disorder is Huntington's disease. In some embodiments, the method improves at least one selected from the group consisting of locomotion, learning and memory, and life expectancy of the subject suffering from Huntington's disease.

In some embodiments, the neurological disorder is Parkinson's disease. In some embodiments, the method improves cognitive performance and/or reduces impulsivity in the subject suffering from Parkinson's disease. In some embodiments, the method does not induce anxiety in the subject suffering from Parkinson's.

In some embodiments, the compound is included in a pharmaceutical composition. In some embodiments, the pharmaceutical composition further includes one or more pharmaceutically acceptable carriers contemplated herein.

In some embodiments, the method further includes administering to the subject an additional therapeutic agent. In some embodiments, the compound and the additional therapeutic agent is co-administered to the subject. In some embodiments, the compound and the additional therapeutic agent are co-formulated. In some embodiments, the compound is administered to the subject a given period of time before or after the additional therapeutic agent is administered to the subject.

In some embodiments, the subject is a mammal, such as a human.

### Combination Therapies

In one non-limiting embodiment, the compounds of the present invention are useful in the methods of present invention in combination with one or more additional compounds useful for treating, ameliorating, and/or preventing a disease or disorder contemplated within the invention, or a complication or symptom thereof. These additional compounds may comprise compounds of the present invention or other compounds, e.g., commercially available compounds, known to treat, prevent, or reduce the symptoms of a disease or disorder contemplated within the invention.

In some embodiments, the one or more additional compounds is administered before, after, or at the same time the compound of the present invention is administered.

In some non-limiting examples, the additional compounds include a dopamine promoter, such as rotigotine, carbidopa/levodopa, entacapone, ropinirole, cabergoline, pramipexole, tolcapone, bromocriptine, or amantadine; an antidepressant, such as rasagiline, or selegiline; a cognition-enhancing medication such as rivastigmine; an anti-tremor medication such as benztropine; an alpha-synuclein neutralizing antibody, such as an antibody that neutralizes alpha-synuclein oligomer or fibril; or other compounds useful for treating, preventing, ameliorating, and/or managing Parkinson's disease.

In some non-limiting examples, the additional compounds include a cognition-enhancing medication, such as memantine, rivastigmine, galantamine, donepezil; an antibody capable of removing the amyloid plaque; or other compounds useful for treating, preventing, ameliorating, and/or managing Alzheimer's disease.

In some non-limiting examples, the additional compounds include tetrabenazine, antipsychotic drugs, antidepressants, tranquilizers, or other compounds useful for treating, preventing, ameliorating, and/or managing Huntington's disease.

In non-limiting examples, the additional compounds include a glutamate blocker, such as riluzole (6-(trifluoromethoxy)benzothiazol-2-amine); a muscle relaxant such as baclofen or tizanidine; ceftriaxone ((6R,7R)-7-{[(2Z)-2-(2-amino-1,3-thiazol-4-yl)->2-(methoxyimino)acetyl]amino}-3-{[(2-methyl-5,6-dioxo-1,2,5,6-tetrahydro-1,2,4-triazin-3-yl)thio]methyl}-8-oxo-5-thia-1-azabicyclo[4.2.0]oct-2-ene-2-carboxylic acid); or other compounds useful for treating, preventing, ameliorating, and/or managing amyotrophic lateral sclerosis (ALS).

A synergistic effect may be calculated, for example, using suitable methods such as, for example, the Sigmoid-Eₘₐₓ equation (Holford & Scheiner, 1981, Clin. Pharmacokinet. 6: 429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22: 27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Pharmaceutical Compositions and Formulations

The instant specification provides pharmaceutical compositions comprising at least one compound of the instant specification or a salt or solvate thereof, which are useful to practice methods of the instant specification. Such a pharmaceutical composition may consist of at least one compound of the instant specification or a salt or solvate thereof, in a form suitable for administration to a subject, or the pharmaceutical composition may comprise at least one compound of the instant specification or a salt or solvate thereof, and one or more pharmaceutically acceptable carriers, one or more additional ingredients, or any combinations of these. At least one compound of the instant specification may be present in the pharmaceutical composition in the form of a physiologically acceptable salt, such as in combination with a physiologically acceptable cation or anion, as is well known in the art.

In some embodiments, the pharmaceutical compositions useful for practicing the method of the instant specification may be administered to deliver a dose of between 1 ng/kg/day and 100 mg/kg/day. In other embodiments, the pharmaceutical compositions useful for practicing the instant specification may be administered to deliver a dose of between 1 ng/kg/day and 1,000 mg/kg/day.

The relative amounts of the active ingredient, the pharmaceutically acceptable carrier, and any additional ingredients in a pharmaceutical composition of the instant specification will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the instant specification may be suitably developed for nasal, inhalational, oral, rectal, vaginal, pleural, peritoneal, parenteral, topical, transdermal, pulmonary, intranasal, buccal, ophthalmic, epidural, intrathecal, intravenous, or another route of administration. A composition useful within the methods of the instant specification may be directly administered to the brain, the brainstem, or any other part of the central nervous system of a mammal or bird. Other contemplated formulations include projected nanoparticles, microspheres, liposomal preparations, coated particles, polymer conjugates, resealed erythrocytes containing the active ingredient, and immunologically-based formulations.

In some embodiments, the compositions of the instant specification are part of a pharmaceutical matrix, which allows for manipulation of insoluble materials and improvement of the bioavailability thereof, development of controlled or sustained release products, and generation of homogeneous compositions. By way of example, a pharmaceutical matrix may be prepared using hot melt extrusion, solid solutions, solid dispersions, size reduction technologies, molecular complexes (e.g., cyclodextrins, and others), microparticulate, and particle and formulation coating processes. Amorphous or crystalline phases may be used in such processes.

The route(s) of administration will be readily apparent to the skilled artisan and will depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology and pharmaceutics. In general, such preparatory methods include the step of bringing the active ingredient into association with a carrier or one or more other accessory ingredients, and then, if necessary or desirable, shaping or packaging the product into a desired single-dose or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the instant specification is contemplated include, but are not limited to, humans and other primates, mammals including commercially relevant mammals such as cattle, pigs, horses, sheep, cats, and dogs.

In some embodiments, the compositions of the instant specification are formulated using one or more pharmaceutically acceptable excipients or carriers. In some embodiments, the pharmaceutical compositions of the instant specification comprise a therapeutically effective amount of at least one compound of the instant specification and a pharmaceutically acceptable carrier. Pharmaceutically acceptable carriers, which are useful, include, but are not limited to, glycerol, water, saline, ethanol, recombinant human albumin (e.g., RECOMBUMIN®), solubilized gelatins (e.g., GELOFUSINE®), and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), recombinant human albumin, solubilized gelatins, suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, isotonic agents, for example, sugars, sodium chloride, or polyalcohols such as mannitol and sorbitol, are included in the composition. Prolonged absorption of the injectable compositions may be brought about by including in the composition an agent that delays absorption, for example, aluminum monostearate or gelatin.

Formulations may be employed in admixtures with conventional excipients, i.e., pharmaceutically acceptable organic or inorganic carrier substances suitable for oral, parenteral, nasal, inhalational, intravenous, subcutaneous, transdermal enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring, and/or fragrance-conferring substances and the like. They may also be combined where desired with other active agents, e.g., other analgesic, anxiolytics or hypnotic agents. As used herein, “additional ingredients” include, but are not limited to, one or more ingredients that may be used as a pharmaceutical carrier.

The composition of the instant specification may comprise a preservative from about 0.005% to 2.0% by total weight of the composition. The preservative is used to prevent spoilage in the case of exposure to contaminants in the environment. Examples of preservatives useful in accordance with the instant specification include but are not limited to those selected from the group consisting of benzyl alcohol, sorbic acid, parabens, imidurea and any combinations thereof. One such preservative is a combination of about 0.5% to 2.0% benzyl alcohol and 0.05-0.5% sorbic acid.

The composition may include an antioxidant and a chelating agent that inhibit the degradation of the compound. Antioxidants for some compounds are BHT, BHA, alpha-tocopherol and ascorbic acid in the exemplary range of about 0.01% to 0.3%, or BHT in the range of 0.03% to 0.1% by weight by total weight of the composition. The chelating agent may be present in an amount of from 0.01% to 0.5% by weight by total weight of the composition. Exemplary chelating agents include edetate salts (e.g. disodium edetate) and citric acid in the weight range of about 0.01% to 0.20%, or in the range of 0.02% to 0.10% by weight by total weight of the composition. The chelating agent is useful for chelating metal ions in the composition that may be detrimental to the shelf life of the formulation. While BHT and disodium edetate are exemplary antioxidant and chelating agent, respectively, for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethyl cellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, acacia, and ionic or non-ionic surfactants. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the instant specification may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the instant specification may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, ionic and non-ionic surfactants, and a preservative. Additional excipients, such as fillers and sweetening, flavoring, or coloring agents, may also be included in these formulations.

A pharmaceutical composition of the instant specification may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying. Methods for mixing components include physical milling, the use of pellets in solid and suspension formulations and mixing in a transdermal patch, as known to those skilled in the art.

### Administration/Dosage/Formulations

The regimen of administration may affect what constitutes an effective amount. The therapeutic formulations may be administered to the patient either prior to or after the onset of the disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, for example a mammal, for example a human, may be carried out using known procedures, at dosages and for periods of time effective to treat the disease or disorder in the patient. An effective amount of the therapeutic compound necessary to achieve a therapeutic effect may vary according to factors such as the state of the disease or disorder in the patient; the age, sex, and weight of the patient; and the ability of the therapeutic compound to treat the disease or disorder in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

In particular, the selected dosage level will depend upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle.

In certain embodiments, the compositions of the invention are formulated using one or more pharmaceutically acceptable excipients or carriers. In certain embodiments, the pharmaceutical compositions of the invention comprise a therapeutically effective amount of a compound of the invention and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils.

In certain embodiments, the compositions of the invention are administered to the patient in dosages that range from one to five times per day or more. In other embodiments, the compositions of the invention are administered to the patient in range of dosages that include, but are not limited to, once every day, every two, days, every three days to once a week, and once every two weeks. It will be readily apparent to one skilled in the art that the frequency of administration of the various combination compositions of the invention will vary from individual to individual depending on many factors including, but not limited to, age, disease or disorder to be treated, gender, overall health, and other factors. Thus, the invention should not be construed to be limited to any particular dosage regime and the precise dosage and composition to be administered to any patient will be determined by the attending physical taking all other factors about the patient into account.

Compounds of the invention for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 3050 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 50 mg to about 1,000 mg, about 75 mg to about 900 mg, about 100 mg to about 800 mg, about 250 mg to about 750 mg, about 300 mg to about 600 mg, about 400 mg to about 500 mg, and any and all whole or partial increments therebetween.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., a drug used for treating the disease or disorder, or a complication or symptom thereof) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of the disease or disorder in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions of the invention include nasal, inhalational, topical, oral, buccal, rectal, pleural, peritoneal, vaginal, intramuscular, subcutaneous, transdermal, epidural, intratracheal, otic, intraocular, intrathecal and intravenous. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the invention may be in the form of tablets or capsules prepared by conventional means with pharmaceutically acceptable excipients such as binding agents (e.g., polyvinylpyrrolidone, hydroxypropylcellulose or methylcellulose, hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

The present invention also includes a multi-layer tablet comprising a layer providing for the delayed release of one or more compounds of the invention, and a further layer providing for the immediate release of a medication for treatment of the disease or disorder. Using a wax/pH-sensitive polymer mix, a gastric insoluble composition may be obtained in which the active ingredient is entrapped, ensuring its delayed release.

### Parenteral Administration

For parenteral administration, the compounds of the invention may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Additional Administration Forms

Additional dosage forms of this invention include dosage forms as described in U.S. Pat. Nos. 6,340,475, 6,488,962, 6,451,808, 5,972,389, 5,582,837, and 5,007,790. Additional dosage forms of this invention also include dosage forms as described in U.S. Patent Applications Nos. 20030147952, 20030104062, 20030104053, 20030044466, 20030039688, and 20020051820. Additional dosage forms of this invention also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation.

In a specific embodiment of the invention, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the present invention will depend on the age, sex and weight of the patient, the current medical condition of the patient and the progression of the disease or disorder in the patient being treated. The skilled artisan will be able to determine appropriate dosages depending on these and other factors.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

The compounds for use in the method of the invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

### Example 1-1

Glutamate is a major excitatory neurotransmitter in the central nervous system which plays a key role in neuroplasticity, cognition, learning, memory, and development. The neurotransmission of glutamate is tightly regulated by a network of metabotropic and ionotropic receptors, and transporters that are distributed on neurons, glia and other cell types in the brain. Under healthy conditions, depolarization of the glutamate neurons leads to the release of glutamate by the presynaptic vesicles in a calcium dependent manner, which then activates the post synaptic ionotropic NMDA and AMPA receptors facilitating the activation of calcium dependent signaling cascades in the post synaptic neuron. Excess glutamate is quickly removed from the synapse by the astrocytic glutamate reuptake transporters which is an active transport involving co-transport of glutamate along with 3 Na⁺ followed by counter transport of K⁺ ion. This removal of excess glutamate from the synapse prevents hyper stimulation of the post synaptic and extra synaptic NMDA receptors to prevent excitotoxicity. In the peri synaptic astrocytes, glutamate is converted into glutamine, by glutamine synthetase and glutamine is recycled back into the neuron, wherein it is converted back to glutamate by glutaminase and then packaged into vesicles by the vesicular glutamate transporter.

The glutamate transporters are classified into 5 subtypes in rodents and humans. The three rodent transporters are called GLAST, GLT1 and EAAC1 which are predominantly transported in the rat brain. The human homologue of these three transporters is called the excitatory amino acid transporters (EAAT) EAAT1, EAAT2 and EAAT3, respectively. The remaining two EAAT4 and EAAT5 share common nomenclature in rodents and humans. These transporters are differentially expressed in various brain regions; GLAST is more prominent in the cerebellum, GLT1 (or EAAT2) is found mostly in forebrain with minimal expression in cerebellum. They are found on the membranes of astroglia or in Bregman glia associated with excitatory synapse. EAAT3 is ubiquitously present in the various brain regions. EAAT4 and EAAT5 are expressed in cerebellum and retina respectively. EAAT2 is the most common subtype and accounts for 85-90% of glutamate reuptake from the terminals. Dysfunction or downregulation of EAAT2 can lead to excess accumulation of glutamate which can not only over activate the post synaptic NMDA and AMPA receptors but also the extrasynaptic NMDA receptors resulting in calcium mediated excitotoxicity and death. This form of excitotoxicity has been implicated in several neurological disorders including ALS, AD, HD and PD and dysfunction of EAAT2 has been documented in these disorders suggesting EAAT2 could be a novel drug target to treat excitotoxicity in these disorders. In fact, Ceftrioxone a β-lactam antibiotic, which has non-specific EAAT2 activator functions was advanced through Phase 3 clinical trials for treatment of ALS. In an effort to develop novel EAAT2 activators, a cell-based enzyme linked immunosorbent assay (ELISA) was developed where primary astrocyte line was stably transfected with designed vector for EAAT2 modulation. This assay was used for high throughput screening of a library of compounds for glutamate reuptake performed using MDCK-hEAAT2 transflex plate assay. Several hits were identified with micromolar efficacy in glutamate reuptake assay using this method. A small molecule activator of EAAT2 was designed and was tested for efficacy in preclinical models of ALS and Epilepsy. However, none of these molecules have progressed to become FDA approved drugs due to either off-target toxicity or lack of translatable physicochemical properties.

A series of small molecule activators of EAAT2 exemplified by GT951 was developed using a hybrid structure-based screening approach (see WO 2018/132829 A1, the entirety of the PCT publication is hereby incorporated by reference). GT951 and its analogs have high selectivity and specificity to EAAT2 with nanomolar efficacy and do not activate postsynaptic NMDA receptors. Site directed mutagenesis studies confirmed that these compounds bind to an allosteric site that is at the interface between the trimerization and transport domain but proximal to the substrate binding domain. In this study, the pharmacokinetic profile of compound GT951 was measured and structure guided medicinal chemistry-based optimization was performed to derive novel molecules GT467 and GT511 and tested their pharmacokinetic profile in rats.

### Example 1-2: Materials and Methods

### Library Design and Molecular Docking

The three-dimensional structure of EAAT2 was modeled using the bacterial GltPh. The binding pocket for GT951 and its analogs were validated using site directed mutagenesis studies and the binding site was shown to be distinct from the substrate translocation domain. Using the pharmacophore derived from GT951, a library of small molecules was screened using the hybrid structure-based screening method to obtain 3822 hit molecules. The molecules were docked to the same binding pocket using GOLD docking software and scored with GoldScore. From the rank ordered molecules, ten best ranking molecules were synthesized and tested using the in vitro transflex plate assay for glutamate uptake.

### Chemicals and Reagents

Common reagents and solvents were purchased from commercial suppliers and used without further purification unless otherwise stated. Compounds (D1-D9) were kindly provided by EDASA Scientific. Tetrahydrofuran (THF) was distilled from sodium-benzophenone under an argon atmosphere. Reaction progress was monitored using analytical thin-layer chromatography (TLC) on precoated silica gel GF254 plates (Macherey Nagel GmbH & Co. KG, Düren, Germany), and spots were detected under UV light (254 and 366 nm). Compounds were purified with flash column chromatography with a silica gel and particle size of 40-63 μM (Merck, Darmstadt, Germany) as the stationary phase and hexane/ethyl acetate or dichloromethane/methanol mixtures as eluent systems. Nuclear magnetic resonance spectra were measured on a Bruker AV-400 NMR instrument (Bruker, Karlsruhe, Germany) in deuterated solvents (DMSO-d6, CDCl3, MeOD-d4). Chemical shifts are expressed in ppm relative to DMSO-d6, CDCl3, or MeOD-d4 (2.50/7.26/3.31 for 1H; 39.52/77.16/49.00 for 13C). The following abbreviations are used to set multiplicities: s=singlet, d=doublet, t=triplet, q=quartet, m=multiplet, br.=broad. IR spectra were recorded on Thermo Nicolet IR-200 in KBr. The mass spectra were recorded on a Finnigan MAT mass spectrometer using electron impact ionization (direct inlet) and an ITD-700 detector. The ionizing electron energy was 70 eV and the mass range was m/z 35-400. Measurements for verification and purity of the compounds were performed by LC/MS. LC-MS/MS data were obtained using a Dionex Ultimate 3000 liquid chromatograph (Dionex, USA) connected to an AB Sciex Qtrap 3200 mass spectrometer (AB Sciex, Canada). LC separation was carried out on a Shim-pack GIST C18-AQ (150 mm×2.1 mm, 3 μm, Shimadzu, Japan) column. Mobile phase consisted of the mixture of 10 mM ammonium formate with the addition of 0.2% (v/v) formic acid in water (A) and acetonitrile (B). The following gradient program was applied: 0.0-1.0 min (5% B), 1.0-7.5 min (5% B-50% B), 7.5-8.5 min (50% B-90% B), 8.5-11.5 min (90% B-90% B), 11.5-12.0 min (90% B-5% B), 12.0-17.0 min (5% B-5% B). The mobile phase flow rate was 0.4 mL min-1. The injection volume was 10 μL. Compounds were detected at λ=254 nm, and target compounds were ≥95% pure. High resolution mass spectra (HRMS) were measured on a Bruker maX is instrument using electrospray ionization (ESI). The melting points were measured in open capillaries and presented without correction. All reactions were performed on a gram-scale.

### Synthesis and Characterization of Compounds

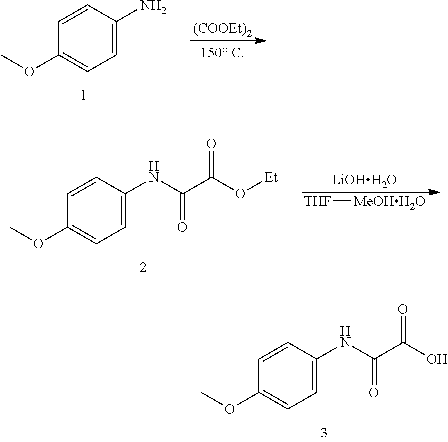

### Ethyl 2-((4-methoxyphenyl)amino)-2-oxoacetate (2)

4-Methoxyaniline (1) (20 g, 162 mmol, 1 equiv.) was added to a solution of diethyl oxalate (88 mL, 649 mmol, 4 equiv.) at ambient temperature. The reaction mixture was stirred at 150° C. for 4 h. After confirming that the reaction was complete by TLC analysis, the resulting solution was cooled to room temperature and poured into 250 mL of hexane. The white precipitate was collected by filtration and dried to afford 30 g of light yellow solid. (Yield: 83%) M.p.=99° C. (lit. mp=100-104° C.)

¹H NMR (400 MHz, CDCl₃): δ=8.83 (br. S., 1 H), 7.59 (d, J=9.2 Hz, 2 H), 6.92 (d, J=8.9 Hz, 2 H), 4.43 (q, J=7.3 Hz, 2 H), 3.83 (s, 3 H), 1.44 (t, J=7.2 Hz, 3 H).

### 2-((4-Methoxyphenyl)amino)-2-oxoacetic acid (3)

Ethyl 2-((4-methoxyphenyl)amino)-2-oxoacetate (2) (11.15 g, 50 mmol, 1 equiv.) was dissolved in MeOH-THF-H₂O (25+25+25 ml) system, then LiOH*H₂O (2.25 g, 55 mmol, 1.1 equiv.) was added in one portion. Reaction mixture was stirred at room temperature for 1 hour, then THF and MeOH were evaporated, water phase was acidified with 10% HCl until pH=1. Precipitate was filtered off, dried in vacuo to afford 8.3 g (85%) of 3 as a white solid of analytical purity with m.p.=206° C. (decomp.) (lit. mp=215° C. (decomp.))

¹H NMR (400 MHz, DMSO-d₆): δ=14.10 (br. S., 1 H), 10.61 (s, 1 H), 7.68 (d, J=8.9 Hz, 2 H), 6.92 (d, J=8.9 Hz, 2 H), 3.73 (s, 3 H).

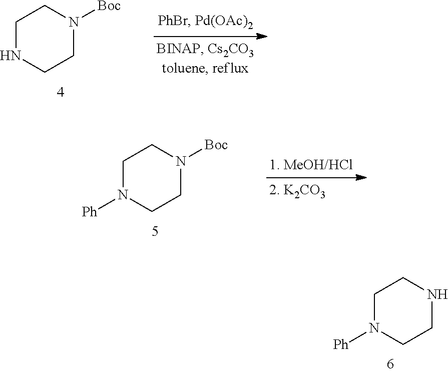 [SMILES: CC(C)(C)OC(=O)N1CCNCC1 ; CC(C)(C)OC(=O)N1CCN(c2ccccc2)CC1 ; c1ccc(N2CCNCC2)cc1]

### tert-Butyl 4-phenylpiperazine-1-carboxylate (5)

A suspension of BINAP (7.5 g, 12.2 mmol, 0.1 equiv) in 125 ml of dry toluene was heated to reflux and cooled to 80° C. Then palladium acetate (1.37 g, 6.1 mmol, 0.05 equiv) was added under argon atmosphere and resulting solution was allowed to stand to cool to room temperature. Then amine (4) (22.78 g, 122 mmol, 1 equiv), bromobenzene (12.76 ml, 122 mmol, 1.2 equiv) and cesium carbonate (80 g, 244 mmol, 2 equiv) were added. The resulting suspension was heated at 80° C. for 20 hours before all the starting amine converted. After completion of reaction, the solvent was evaporated under vacuum and resulting mixture was separated by flash chromatography using mixture of hexane-ethyl acetate (3:1, Rf=0.4) as eluent to afford 20.8 g (65%) of (5) as a white solid of analytical purity with m.p.=95-97° C. (lit mp=100-104° C.).

¹H NMR (400 MHz, CDCl₃): δ=7.27-7.32 (m, 2 H), 6.94 (d, J=7.9 Hz, 2 H), 6.90 (t, J=9.02 Hz, 1 H), 3.59 (t, J=5.2 Hz, 4 H), 3.14 (t, J=5.01 Hz, 4 H), 1.49 (s, 9 H).

### 1-Phenylpiperazine (6)

Starting carbamate (5) (20.8 g, 80 mmol) was dissolved in MeOH-HCl (1M, 5 equiv., 400 ml). Reaction mixture was stirred at room temperature for 6 hours, then solvent was evaporated. Residue was dissolved in water (200 ml), then basified with K₂CO₃ until pH=9. After that, water was evaporated, residue was treated with hot isopropyl alcohol (3×150 ml), precipitate was filtered off and washed twice with isopropyl alcohol. Then, solvent was evaporated to afford 9.8 g (76%) of (6) as a yellow oil.

¹H NMR (400 MHz, CDCl₃): δ=7.27-7.33 (m, 2 H), 6.91-6.98 (m, 3 H), 5.22 (br. S., 1 H), 3.31 (t, J=4.92 Hz, 4 H), 3.21 (t, J=4.92 Hz, 4 H).

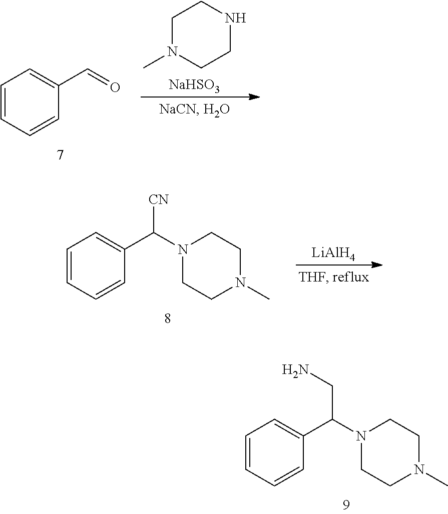

### 2-(4-Methylpiperazin-1-yl)-2-phenylacetonitrile (8)

Sodium bisulfite (26 g, 250 mmol, 1 equiv.) was dissolved in water (250 ml), benzaldehyde (7) (25.5 ml, 250 mmol, 1 equiv.) was added, the mixture was heated to 60° C., then cooled to 35° C. N-methyl piperazine (27.78 ml, 250 mmol, 1 equiv.) was added in one portion and mixture was stirred at room temperature for 2 hours. Then solution of sodium cyanide (12.25 g, 250 mmol) in water (250 ml) was added with vigorous stirring. After 90 minutes, water phase was extracted with CH₂Cl₂ (3×200 ml), organic phase was washed with brine, dried over anhydrous Na₂SO₄ and solvent was evaporated. Residue was purified using flash-chromatography (EtOAc, Rf=0.2) to afford 47.82 g (89%) of 8 as a yellow solid of analytical purity with m.p.=66° C. (lit mp=67-69° C.).

¹H NMR (400 MHz, CDCl₃): δ=7.53 (d, J=7.0 Hz, 2 H), 7.32-7.44 (m, 3 H), 4.84 (s, 1 H), 2.35-2.79 (m, 8 H), 2.29 (s, 3 H).

### 2-(4-Methylpiperazin-1-yl)-2-phenylethan-1-amine (9)

LiAlH₄ (42.18 g, 5 equiv., 1.11 mol) was added to a THE solution (1 L), the mixture was cooled to 0° C., the solution of the starting nitrile (8) (47.8 g, 222 mmol) in THF (250 ml) was added dropwise at 0° C. After addition of all the nitrile, the mixture was refluxed for 12 hours, then it was cooled to 0° C. and decomposed by sequential addition of 42.2 ml of water, 42.2 ml of 15% NaOH, 126.6 ml of water. The mixture was filtered, the precipitate was washed with THE, the filtrate was evaporated. The residue was purified using flash-chromatography (DCM:MeOH (NH₃), Rf=0.2) to afford 10.9 g (26%) of 9 as a yellow oil.

¹H NMR (400 MHz, CDCl₃): δ=7.29-7.36 (m, 3 H), 7.21-7.28 (m, 2 H), 3.31 (t, J=6.3 Hz, 1 H), 3.06-3.14 (m, 1 H), 2.93-3.02 (m, 1 H), 2.34-2.64 (m, 10 H), 2.25 (s, 3 H).

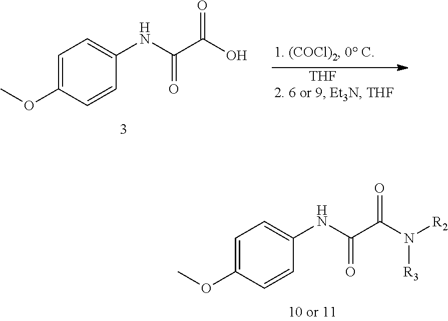

### General Procedure for Amidization

Starting acid (3) (1.1 equiv.) was dissolved in THF (1M solution), drop of DMF was added and solution was cooled to 0° C. Oxalyl chloride (1.2 equiv.) was added drop by drop at this temperature, then reaction mixture was stirred at room temperature for 1 hour. Solvent was evaporated, then residue was dissolved in THF (1M solution). This solution was added drop by drop to the solution of appropriate amine 6 or 9 (1 equiv.) and Et₃N (3 equiv.) in THF (1M solution) at 0° C. After addition, reaction mixture was stirred overnight. THF was evaporated, residue was treated with water, water phase was extracted with CH₂Cl₂, organic phase was washed with water, with brine and dried over anhydrous Na₂SO₄. Solvent was evaporated, residue was purified using flash-chromatography.

### N-(4-Methoxyphenyl)-2-oxo-2-(4-phenylpiperazin-1-yl)acetamide (10)

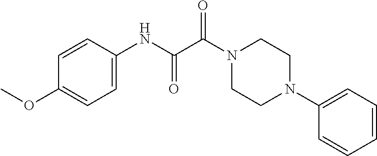 [SMILES: COc1ccc(NC(=O)C(=O)N2CCN(c3ccccc3)CC2)cc1]

This compound was made using procedure described above, eluent: Hex:EtOAc (3:1, Rf=0.3). Yield: 45% (1.02 g) as a white solid of analytical purity with m.p.=174-178° C.

¹H NMR (400 MHz, DMSO-d₆): δ=9.15 (br. S., 1 H), 7.53 (d, J=9.05 Hz, 2 H), 7.30 (t, J=8.02 Hz, 2 H), 6.86-6.99 (m, 5 H), 4.46 (t, J=5.13 Hz, 4 H), 3.89 (t, J=5.2 Hz, 4 H), 3.82 (s, 3 H), 3.28 (t, J=4.92 Hz, 4 H).

¹³C NMR (100 MHz, DMSO-d₆): δ=162.64, 161.25, 155.93, 150.68, 130.97, 129.01 (2 C), 121.30 (2 C), 119.61, 116.10 (2C), 113.91 (2C), 55.17, 48.90, 48.33, 45.55, 40.87.

HRMS (ESI): for C₁₉H₂₁N₃O₃ [M+H]⁺: calcd 340.1655, found 340.1660.

### N¹-(4-Methoxyphenyl)-N²-(2-(4-methylpiperazin-1-yl)-2-phenylethyl)oxalamide (11)

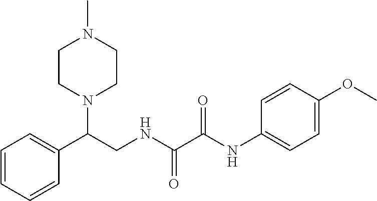 [SMILES: COc1ccc(NC(=O)C(=O)NCC(c2ccccc2)N2CCN(C)CC2)cc1]

This compound was made using procedure described above, eluent: DCM:MeOH (10:1, Rf=0.2). Yield: 43% (960 mg) as a white solid of analytical purity with m.p.=252-256° C.

¹H NMR (400 MHz, DMSO-d₆): δ=10.53 (s, 1 H), 8.54 (t, J=5.5 Hz, 1 H), 7.70 (d, J=9.11 Hz, 2 H), 7.20-7.37 (m, 5 H), 6.89 (d, J=9.03 Hz, 2 H), 3.65-3.78 (m, 5 H), 3.47-3.55 (m, 1 H), 2.16-2.46 (m, 8 H), 2.09 (s, 3 H).

¹³C NMR (100 MHz, DMSO-d₆): δ=164.12, 159.76, 157.91, 156.08, 136.75, 130.69, 128.61 (2C), 128.03 (2C), 127.51, 121.82 (2C), 113.82 (2C), 66.67, 55.21, 54.55, 44.89, 40.19.

HRMS (ESI): for C₂₂H₂₈N₄O₃ [M+H]⁺: calcd 397.2234, found 397.2237.

### Glutamate Reuptake Assay

The glutamate uptake assay was performed using the BioIVT's kit with their customized Transflex™ plate with a 96 well Millipore insert plate seeded with MDCK cells overexpressing either the hEAAT2 or mock GFP. The assay was performed according to the manufacturer's package insert. Briefly, the transflex assay plate was sterilized and placed into a 37° C. 5% CO₂ sterile culture incubator for 20-28 hours prior to performing the assay.

The Hank balanced salt solution (HBSS-0.14M NaCl, 0.005M KCl, 0.001M CaCl₂, 0.0004M MgSO₄, 0.0005M MgCl₂. 6H₂O, 0.0003M Na₂HPO₄.7H₂O, 0.0004M KH₂PO₄, 0.006M D-glucose, 0.004M NaHCO₃) buffer was prepared with Ca²⁺ and Mg²⁺ (pH 7.2-7.4) and prewarmed at 37° C. before use. HBSS was added to the 96 well plate (insert plate) after removing media and incubated at 37° C. for 5 min.

Dosing block plate was prepared by adding 300 ul of 2% DMSO in HBSS (DMSO control) with and without the addition of glutamate or 2% DMSO in HBSS+L-Cysteic acid (1 mM) or experimental compounds (D1-D9) at 1 uM, 3 uM and 10 uM concentration and known positive control GT951 and a Reference Inhibitor (RI) in HBSS+1 μM Glutamic acid. The insert plate was removed from the incubator and placed inside the dosing block plate and the entire setup was placed back in the incubator for 5 min. The insert plate was then removed and placed into a tray containing ice cold PBS (pH-7.4, 1 μM CaCl₂, 1 μM MgCl₂.6H₂O, 1 μM MgSO₄) and the cells were washed 4 times with 150 ul of ice-cold PBS to remove any excess compound or glutamate. The insert plate was removed and placed in a fresh empty dosing plate and 100 ul of extraction solution (Acetonitrile (I):H₂O::50:50) was added and the set up was placed in a shaker bath for 15 min. Finally, the insert plate was centrifuged, and the supernatants and pellets were collected for analysis of glutamate content using LCMS.

Analytical quantification of glutamate from the samples: To 50 μl of sample, 25 μL 100 mM sodium carbonate and 25 μl of 5% v/v benzoyl chloride IACN were added. After 30 min incubation at 4 C, samples were centrifuged for 10 min and the supernatant transferred to LCMS vials. The samples were analyzed on an Acquity I-Class UPLC system coupled to a Synapt G2Si HDMS mass spectrometer in positive ion mode with a heated electrospray ionization (ESI) source in a Z-spray configuration. LC separation was performed on a Waters Acquity UPLC BEH 1.7 μm 2.1×50 mm column using an 0.6 mL/min isocratic solvent flow of A/B 95/5 for 6 minutes followed by washing and reconditioning the column. Eluent A is 0.1% v/v formic acid in water and B is 0.1% v/v formic acid in acetonitrile. Conditions on the mass spectrometer were as follows: capillary voltage 0.5 kV, sampling cone 40, source offset 80, source 120° C., desolvation temperature 250° C., cone gas 0, desolvation gas 1000 L/hr, nebulizer 6.5 bar. The analyzer was operated in resolution mode. Low energy collection between 100 and 1500 Da at 0.2 sec scan time. MSe collected using a ramp trap collision energy 20-40 V. Masses were extracted from the TOF MS TICs using an abs width of 0.05 Da. Data was analyzed using Waters MassLynx and Waters Unifi. A calibration curve of an authentic glutamate standard was used for quantification.

### Animals

Pharmacokinetics (PK) study for GT951 was conducted at Melior Discovery Services (Exton, PA). Fifteen male CD-1 mice 23-50 g was used for the study and the mice were housed 3 per cage. PK studies for GT467 and GT511 were conducted at Piramal Pharma solutions under approved protocols (Pune, India). One hundred and sixty-eight male Wistar rats were used for the study and housed 4 per cage. All animals were maintained on a standard 12 h light cycle and given free access to water and standard rodent chow. Food and water were available ad libitum. Animals were not fasted for any portion of the study and animals were acclimated to the animal care facility for seven days prior to the start of the experiment. The holding room and procedure room temperature was 21-23° C. and humidity was at 40-45% during the experiment (within the normal range of the care facility). All procedures were completed according to Institutional Animal Care and Use Committee (IACUC) approved protocols for housing and for PK studies.

### Pharmacokinetics Evaluation

For GT951 studies, the mice were dosed with 10 mg/kg of GT951 via intraperitoneal (i.p) route and mice (n=3/time point) were sacrificed at 0.5, 1, 3, 5 and 8 h time points and blood and brain samples were collected. Blood was collected prior to euthanasia via retro-orbital vein into heparinized tubes and brain samples were flash frozen on dry ice and stored at −80° C. until further use. Samples were extracted using an acetonitrile/protein precipitation method. Samples were prepared by adding internal standard (e.g. propranolol), vortexed, and protein precipitates centrifuged at 2500 g for 30 minutes. Fifty μL of supernatant was combined with 500 μL of 1:1:1 0.1% formic acid:acetonitrile:water. This sample was analyzed by LC/MS/MS. Levels were determined by extrapolating the concentration from a standard curve using linear regression analysis. Data are expressed as mean±s.e.m.

### Pharmacokinetics Evaluation of GT467 and GT511

Pharmacokinetics (PK) parameters for GT467 and GT511 were evaluated at 10 mg/kg dose (i.p and p.o) and 5 mg/kg dose (i.v) in rats (n=4/time point/drug). Rats were dosed with the drug and blood and brain samples were collected at 0.5, 1, 2, 4, 8 and 24 h time points. Blood was collected via the sub-lingual vein under anesthesia into tubes containing Na₂EDTA as an anticoagulant. Plasma fraction was separated by centrifuging the samples at 10,000 rpm for 5 min and the plasma was transferred to a cryo tube and stored at −80 C. Animals were euthanized and the brain was rapidly removed on ice and weighed. Brain was homogenized in Milli Q water and the homogenate was stored at −80 C until further use. Bioanalysis of the samples was performed using the SCIEX QTRAP 5500 LCMS/MS system with Phenomenex synergy C18 column and carbamazepine as internal standard. A methanol extraction procedure was used to precipitate the proteins in the sample. In addition to the internal standards, drug free plasma and brain samples were prepared for calibration and quantification standards. All samples were within the detectable range of the standard curves. 50 μl of the sample was added to 200 μl of the working solution of the internal standard and centrifuged for 5 min at 10,000 rpm. 60 μl of the supernatant was added to 140 μl of water and mixed well prior to transferring them for quantitative analysis on LCMS.

### Example 1-3: GLT-1 Modulators Dose Dependently Increase Glutamate Uptake in MDCK Cells That Express GLT-1

Using the pharmacophore derived from GT951, a library of small molecules was designed by combinatorial chemistry method and 3822 molecules were modeled. The molecules were docked to the same binding pocket using GOLD docking software and scored with GoldScore. From the rank ordered molecules, ten best ranking molecules were synthesized: GT247 (D1), GT251 (D2), GT467 (D3), GT477 (D4), GT487 (D5), GT511 (D6), GT559 (D7), GT562 (D8), GT619 (D9).

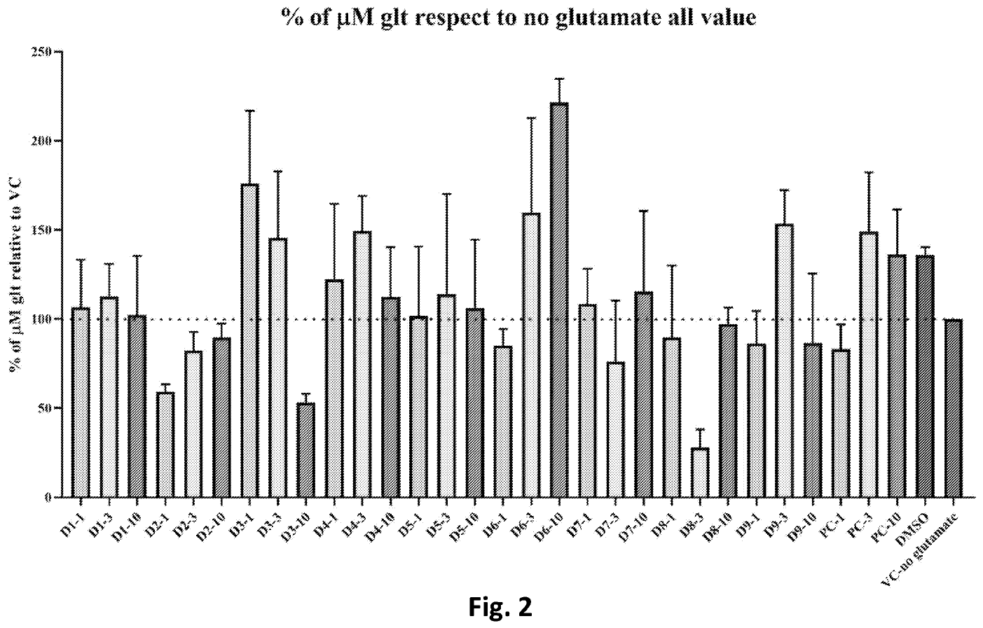

These compounds were tested using the in vitro transflex plate assay for glutamate uptake. In the transflex plate assay, MDCK cells that express GLT-1 were treated with three different concentrations (1 μM, 3 μM and 10 μM) of the compounds, and the glutamate uptakes were measured. The results are shown in Table 1 below, and depicted in FIG. 2:

TABLE 1

| | | % Glutamate level | | | | | |
|---|---|---|---|---|---|---|---|
| | Molecular | 1 μM | | 3 μM | | 10 μM | |
| Compound | Weight | Mean | SEM | Mean | SEM | Mean | SEM |
| GT247 (D1) | 463.54098 | 106.5 | 26.78 | 112.6 | 18.27 | 102.3 | 33.14 |
| GT251 (D2) | 411.50813 | 59.44 | 4.038 | 82.39 | 10.46 | 89.60 | 7.853 |
| GT467 (D3) | 396.49346 | 176.1 | 40.78 | 145.5 | 37.33 | 53.25 | 4.867 |
| GT477 (D4) | 566.66843 | 122.3 | 42.41 | 149.5 | 19.45 | 112.3 | 28.02 |
| GT487 (D5) | 468.60399 | 101.8 | 36.80 | 114.0 | 56.15 | 106.1 | 38.43 |
| GT511 (D6) | 339.39752 | 85.08 | 9.360 | 159.8 | 52.95 | 221.5 | 13.37 |
| GT559 (D7) | 451.57631 | 108.4 | 19.89 | 76.12 | 34.09 | 115.3 | 45.34 |
| GT562 (D8) | 541.70218 | 89.59 | 40.40 | 28.04 | 10.10 | 97.27 | 9.101 |
| GT619 (D9) | 544.66207 | 86.24 | 18.37 | 153.5 | 18.88 | 86.42 | 39.13 |
| GT951 (PC) | 626.07 | 83.28 | 13.78 | 149.0 | 33.28 | 136.4 | 25.08 |

Several compounds were chosen to further test the EC₅₀ for glutamate uptake. The glutamate uptake results for these compounds, as well as the parent compound GT951 (PC) are shown in Table 2 below:

TABLE 2

| Compounds | EC₅₀ (nM) for glutamate uptake |
|---|---|
| GT467 | 35.1 ± 1.0 |
| GT477 | Unstable |
| GT511 | 3.8 ± 2.2 |
| GT559 | 9.3 ± 1.4 |
| GT619 | 78.5 ± 1.0 |
| PC | 11.4 ± 1.3 |

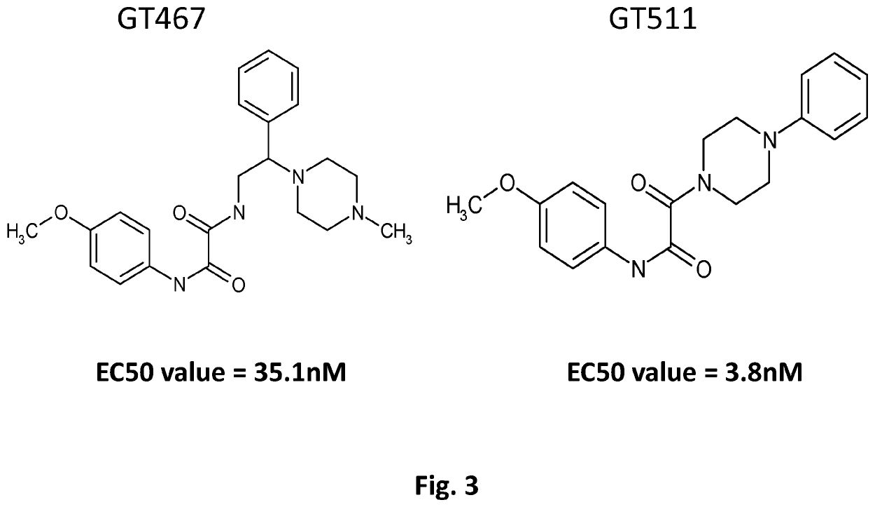

The results demonstrate that several compounds dose dependently increase glutamate uptake in the MDCK cells. Compounds GT467 (D3) and GT511 (D6) (FIG. 3) were chosen for further pharmacokinetics and animal tests.

### Example 1-4: Pharmacokinetic Profiles of GLT-1 Modulators

The present study then tested pharmacokinetic profile of GT511 and GT467 under various routes of administration in rats.

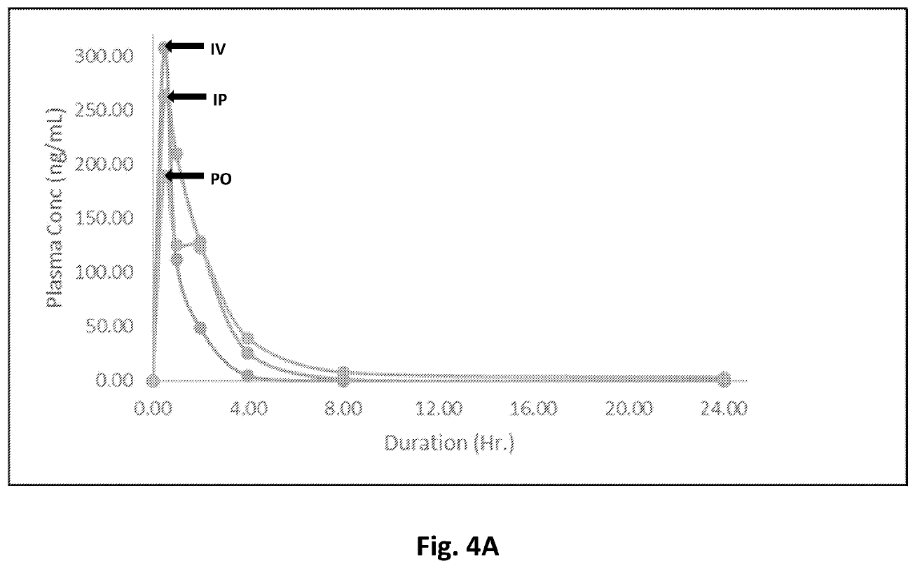

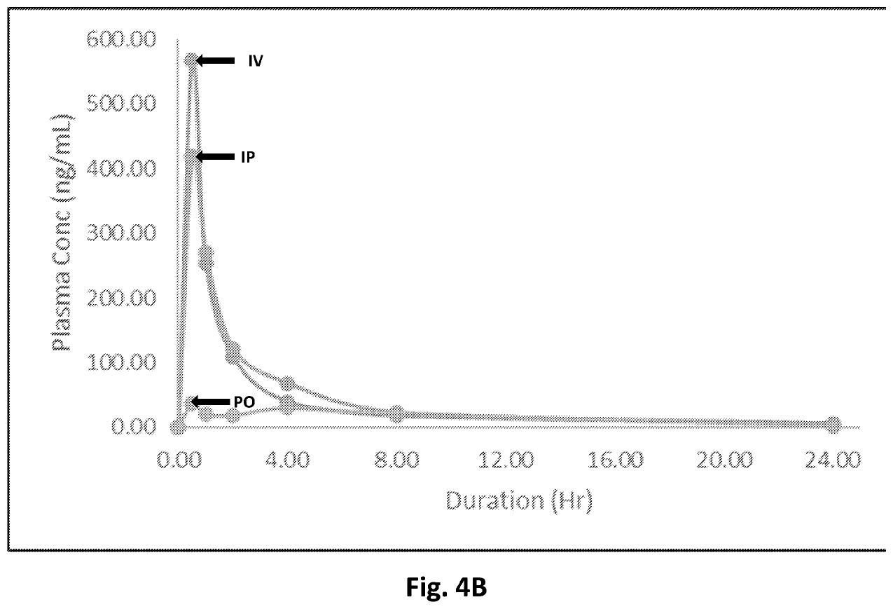

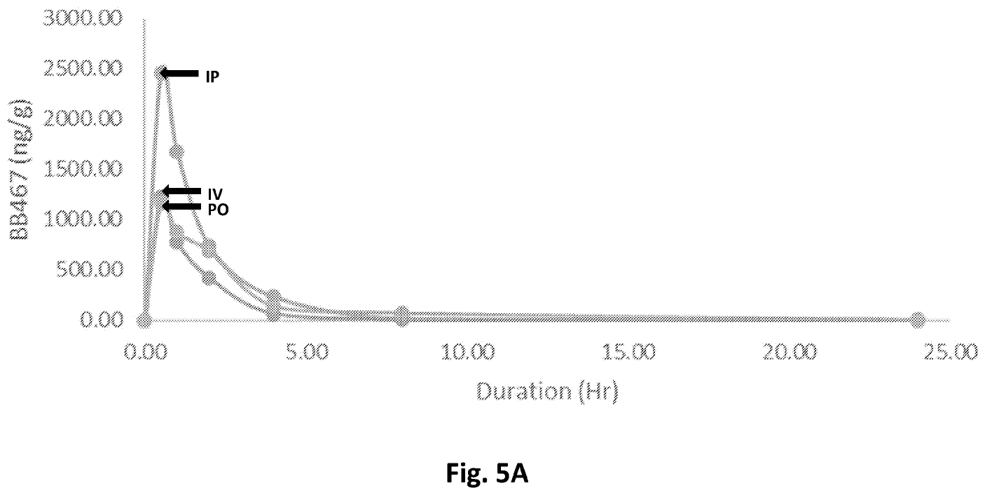

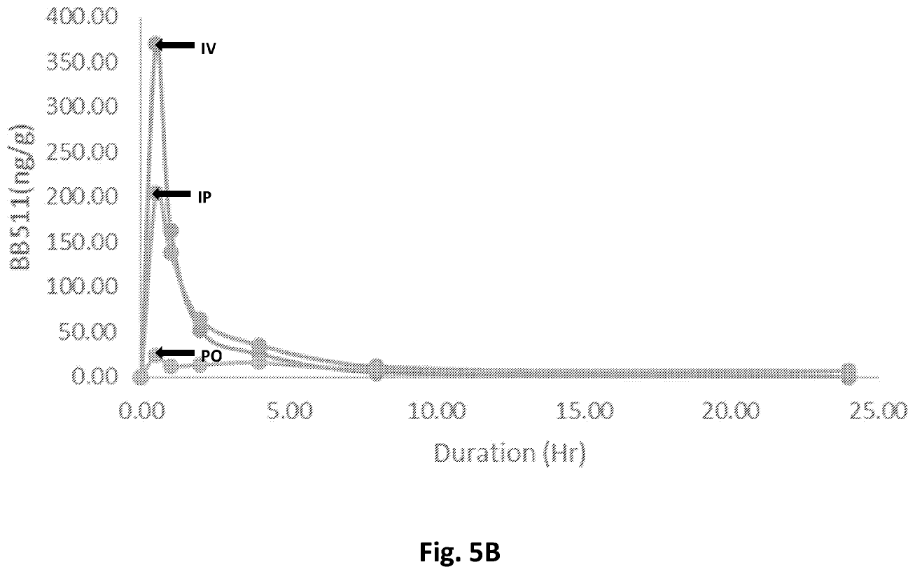

Specifically, rats were administered with GT467 or GT511 intraperitoneally (IP), intravenously (IV) or orally (PO). The concentration of the administered compounds measured at various timepoints in the plasma are depicted in FIGS. 4A and 4B, the concentrations of the administered compounds measured at various timepoints in the brain are depicted in FIGS. 5A and 5B. The results show that, for GT467, all three types of administration methods would result in high plasma concentration and brain concentration. For GT511, intraperitoneal and intravenous administration would result in higher plasma concentration and brain concentration than oral administration.

Furthermore, pharmacokinetic parameters such as the peak concentration (Cₘₐₓ), the time to reach the peak concentration (Tₘₐₓ), the area under curve from time 0 to T (i.e., 24 hrs) (AUC₀₋ₜ), the area under curve from time 0 to infinite (AUC_{0-∞}), the half-life (t_{1/2}), the oral clearance (CL/F), the volume of distribution of drug observed (Vz_F_obs), and the percentage of bioavailability (% Bioavailability) were also measured or calculated. The parameters are listed in Tables 3-6 below:

TABLE 3
Mean pharmacokinetic parameters of GT467 in
plasma following IV IP and PO administration

| Parameter | Unit | IV | IP | PO |
|---|---|---|---|---|
| Cₘₐₓ | ng/g | 307.055 | 262.370 | 204.40 |
| Tₘₐₓ | Hr. | 0.500 | 0.500 | 0.875 |
| AUC₀₋ₜ | hr · ng/ml | 315.635 | 536.768 | 507.172 |
| AUC_{0-∞} | hr · ng/ml | 321.297 | 555.790 | 797.848 |
| t_{1/2} | Hr. | 0.651 | 1.016 | 4.764 |
| CL/F | ml/hr/kg | 16390.725 | 18146.412 | 17914.405 |
| Vz_F_obs | ml/kg | 16170.403 | 26470.969 | 95358.274 |
| % Bioavailability | — | — | 85.03 | 80.34 |

TABLE 4
Mean pharmacokinetic parameters of GT467 brain
content following IV IP and PO administration

| Parameter | Unit | IV | IP | PO |
|---|---|---|---|---|
| Cₘₐₓ | Ng/g | 1221.05 | 2461.17 | 1173.13 |
| Tₘₐₓ | Hr | 0.50 | 0.50 | 0.88 |
| AUC₀₋ₜ | hr · ng/g | 1959.70 | 4414.35 | 2816.80 |
| AUC_{0-∞} | hr · ng/g | 2015.59 | 4442.19 | 3080.18 |
| t_{1/2} | Hr | 1.03 | 1.62 | 1.81 |

TABLE 5
Mean pharmacokinetic parameters of GT511 in
plasma following IV IP and PO administration

| Parameter | Unit | IV | IP | PO |
|---|---|---|---|---|
| Cₘₐₓ | ng/g | 567.22 | 450.27 | 42.86 |
| Tₘₐₓ | hr. | 0.50 | 0.63 | 3.25 |
| AUC₀₋ₜ | hr · ng/ml | 919.43 | 1073.21 | 321.93 |
| AUC_{0-∞} | hr · ng/ml | 995.17 | 1130.71 | 399.24 |
| t_{1/2} | Hr. | 6.08 | 6.22 | 5.34 |
| CL/F | ml/hr/kg | 5178.58 | 8924.85 | 30308.07 |
| Vz_F_obs | ml/kg | 41463.89 | 79221.20 | 198882.89 |
| % Bioavailability | — | — | 58.36 | 17.51 |

TABLE 6
Mean pharmacokinetic parameters of GT511 brain
content following IV IP and PO administration

| Parameter | Unit | IV | IP | PO |
|---|---|---|---|---|
| Cₘₐₓ | Ng/g | 370.60 | 204.10 | 24.98 |
| Tₘₐₓ | Hr | 0.50 | 0.50 | 1.38 |
| AUC₀₋ₜ | hr · ng/g | 443.23 | 429.85 | 183.00 |
| AUC_{0-∞} | hr · ng/g | 483.13 | 467.11 | —* |
| t_{1/2} | Hr | 1.52 | 2.26 | —* |

### Example 1-5: GLT-1 Modulators Improve Locomotion in a Drosophila Model of Huntington's Disease

The present study then evaluated the ability of the GLT-1 modulators to rescue locomotive impairments in a Drosophila model of Huntington's disease (also referred to as “Htt model”).

The Drosophila Htt model is produced by crossing UAS-Htt(128Q) male flies with elav-Gal4 virgin female flies. Both fly strains were purchased from Bloomington Drosophila Stock Center (BDSC, Indiana, USA).

Huntingtin(128Q) (“Htt(128Q)”) is a mutant human gene whose expression produces full-length human Huntingtin having the abnormally long 128 glutamine repeats, which causes Huntington's disease in humans.

In the resulting UAS-Htt(128Q) X elav-Gal4 progeny flies, GAL4 protein expression is driven by the pan-neuronal elav promoter in the neurons of the flies. The GAL4 in the neurons binds to the UAS sequence and drives the expression of Htt(128Q), which results in the neuron specific expression of the mutant Huntingtin protein.

To validate the expression of Htt(128Q) human transgene in neurons of the UAS-Htt(128Q) X elav-Gal4 flies, RT-qPCR was performed. Brains were dissected from the elav-Gal4 X UAS-Htt(128Q) progeny larvae. For control, brains were dissected from larvae expressing elav-Gal4 with wild type strain (w1118). Following RNA extraction and cDNA conversion, real time PCR was performed using primers targeted to the Htt(128Q) transgene as well as reference primers to housekeeping gene RP49. Only the progeny larvae expressing both elav-Gal4 and UAS-Htt(128Q) showed amplification with the Htt(128) primers, therefore validating expression of Htt(128Q) in the Drosophila Huntington's disease model (HD model).

Locomotor ability is significantly reduced in the Drosophila HD model when compared to wild type. Therefore, different compounds can be tested at varying concentrations to test if one or more concentration is neuroprotective.

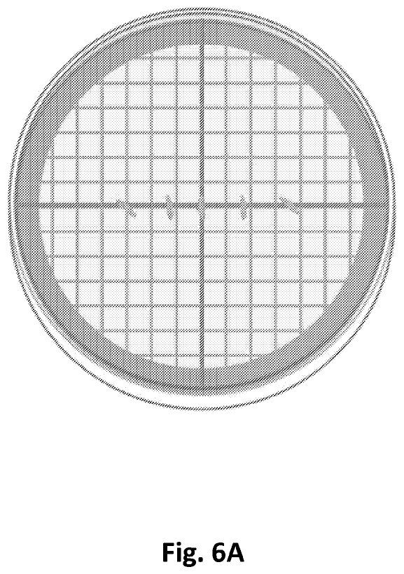

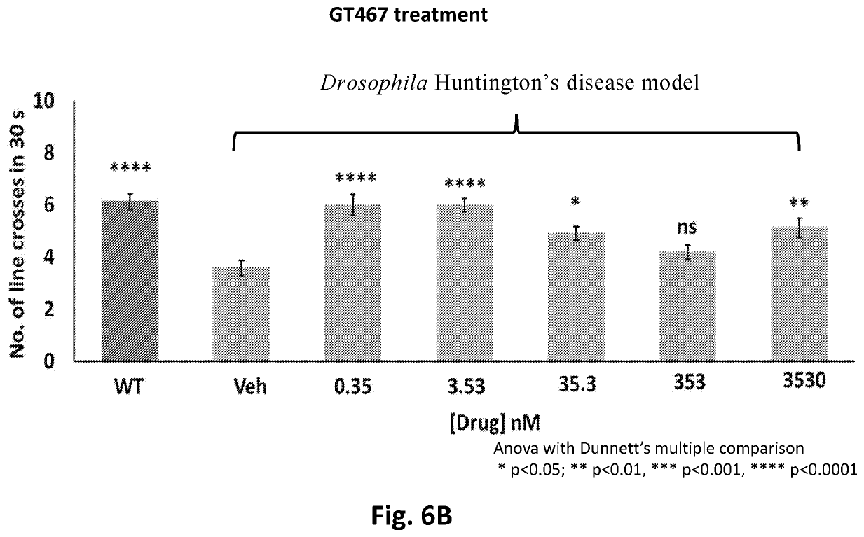

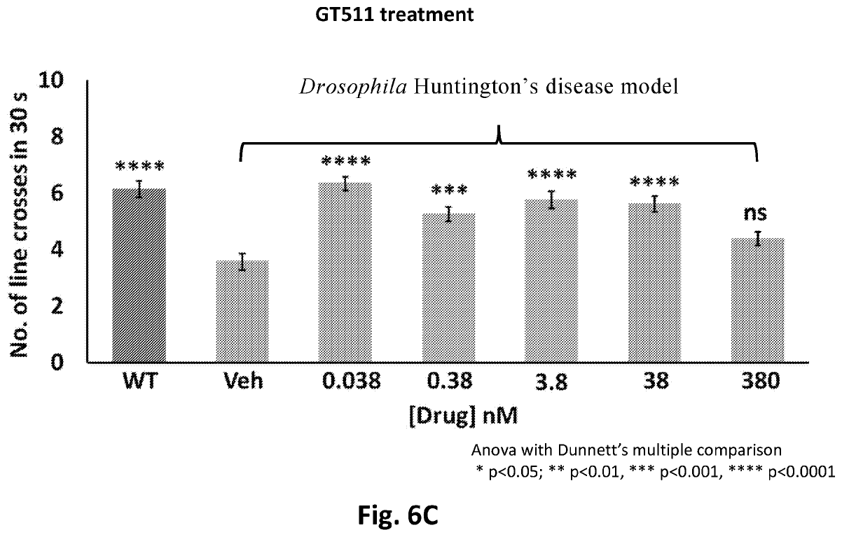

To assess for rescue in locomotive impairments in the Drosophila model of Huntington's Disease (Htt model) by GT467 and GT511, the EC₅₀ of each compound was determined in order to set up five concentrations: two-fold below EC₅₀, one-fold below EC₅₀, at EC₅₀, one-fold above EC₅₀, and two-fold above EC₅₀. Locomotion assays were conducted by transferring the third instar larvae of flies to agar plates (FIG. 6A) and counting the number of grid lines crossed in 30 seconds. The locomotion protocol was repeated until a sample size of 20 larvae was achieved for each compound at five different concentrations.

Referring to FIGS. 6B and 6C (WT: Elav x elav+vehicle, Veh: Elav x HTT+vehicle, Numbers: Elav x HTT+compounds at the indicated concentration), the treatment with both GT467 and GT511 restores the Huntington's disease model flies' locomotor ability. As shown in the figures, these compounds, at concentrations as low as two-fold below EC₅₀ and one-fold below EC₅₀, are able to fully restore the locomotor ability in the Huntington's disease model flies.

### Example 1-6: GLT-1 Modulators Improve Learning and Memory in the Drosophila Model of Huntington's Disease

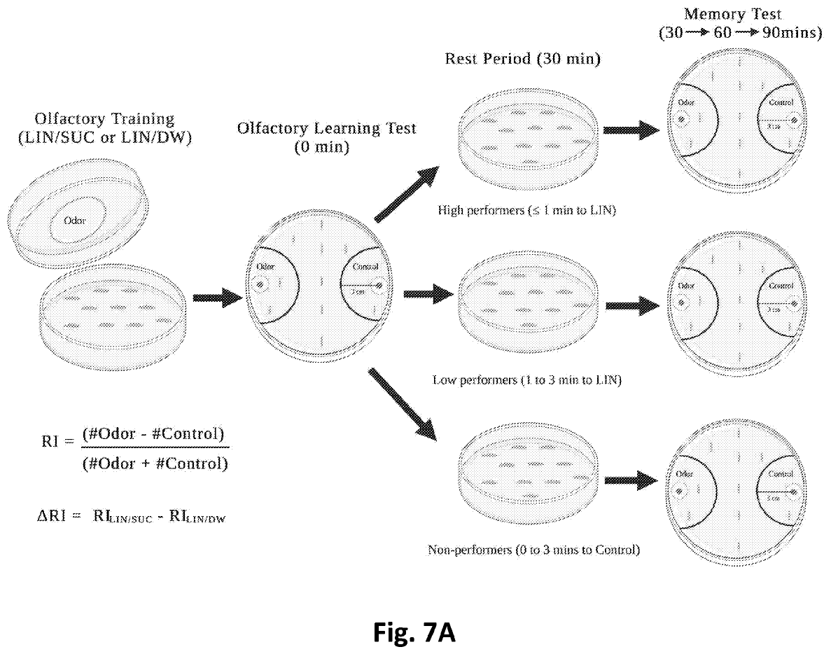

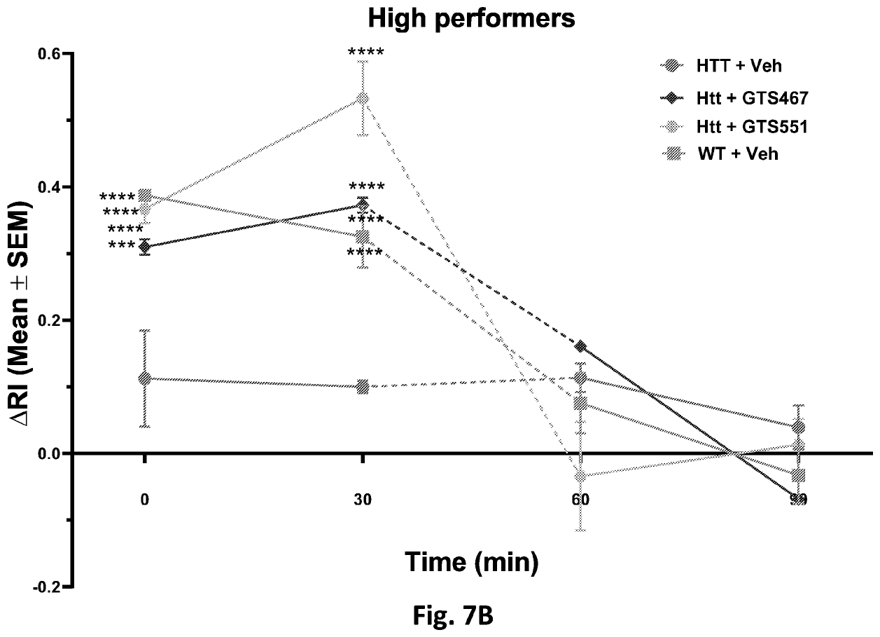

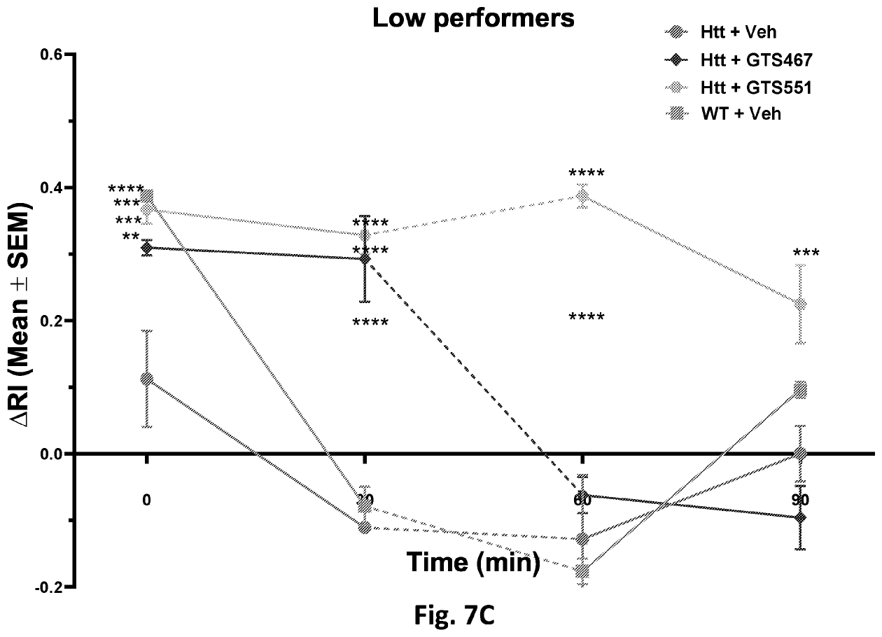

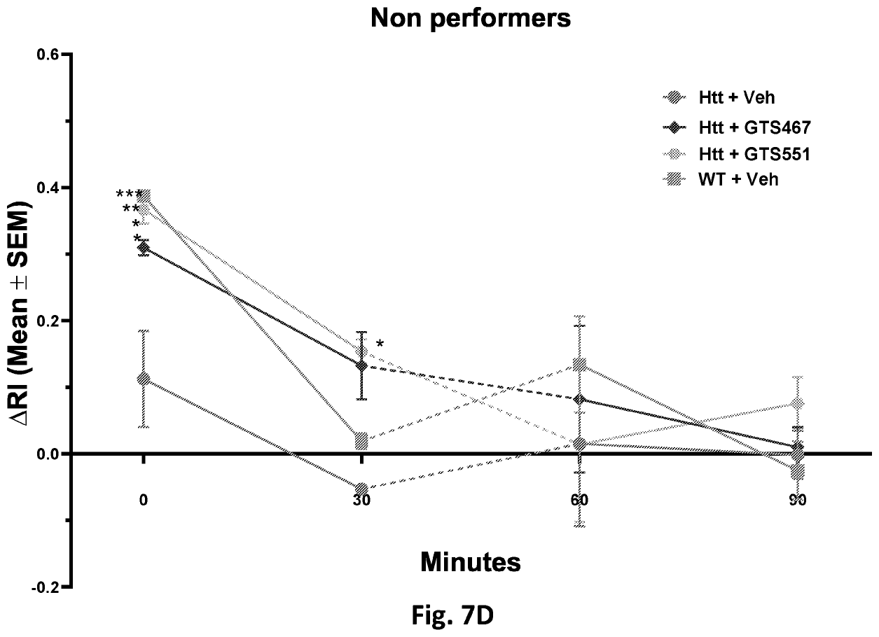

Referring to FIGS. 7A-7D, five groups of Drosophila, “WT+Vhe (Elav x elav treated with vehicle), “Htt+Veh” (Elav x Htt treated with vehicle), “Htt+GT467” (Elav x Htt treated with 3.53 nM of GT 467), and “Htt+GT551” (Elav x Htt treated with 0.038 nM of GT 511), were tested for the learning and memory in the olfactory learning test.

Referring to FIG. 7A, Drosophila larvae were trained with either a reinforcer solution containing sucrose (“LIN/SUC”) or a control of distilled water (“LIN/DW”). The trained larvae were then transferred to a dish having test odorant placed on one side and a control on the opposite side. The ΔRI were then calculated for the high performers (FIG. 7B), low performers (FIG. 7C) and non-performers (FIG. 7D). The results show that the performances of the larvae of the Huntington's disease model performed worse than the performance of the wild-type counterpart, but were improved significantly by treatment with GT 467 and GT 511.

### Example 1-7: GLT-1 Modulator Improves the Longevity in the Drosophila Model of Huntington's Disease

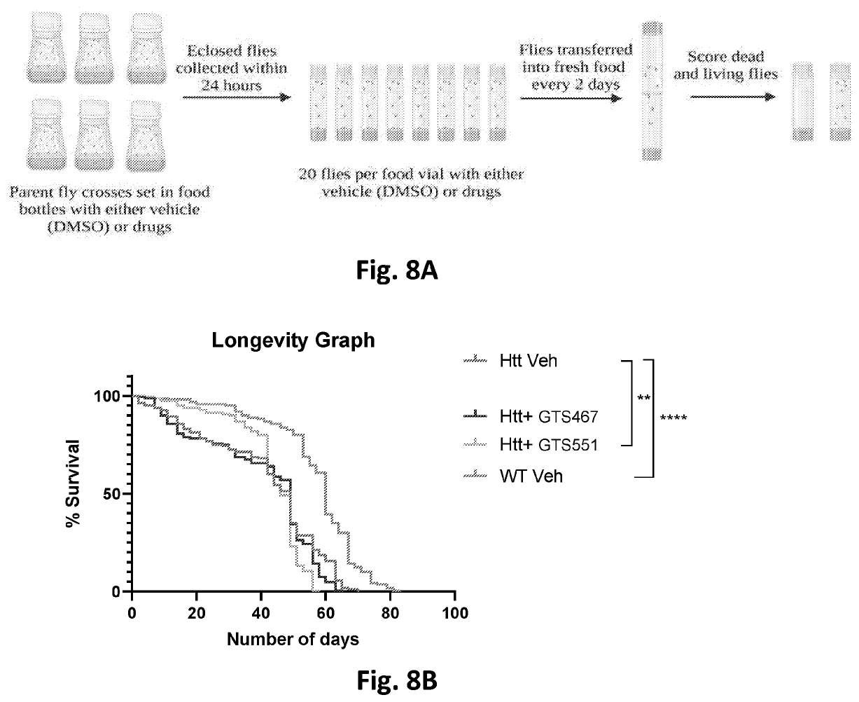

Referring to FIGS. 8A-8B, the present study tested the effect of the exemplary GLT-1 modulators on the longevities of Drosophila of the model of Huntington's disease.

Referring to FIG. 8A, Drosophila flies (either Htt or wildtype) were enclosed in food vials containing either vehicle (DMSO) or the exemplary GLT-1 modulators (GT467, GT511). The survival rates of the flies were recorded over time.

Referring to FIG. 8B, neither of the two exemplary GLT-1 modulators had negative impacts on the longevities of the flies. One of the two exemplary GLT-1 modulators, GT511, significantly improved the survival rate of Drosophila flies of the model of Huntington's disease up to about day 40.

### Example 1-8: GLT-1 Modulator Rescued Locomotor Deficits and Improved Memory in a Mouse Model of Huntington's Disease

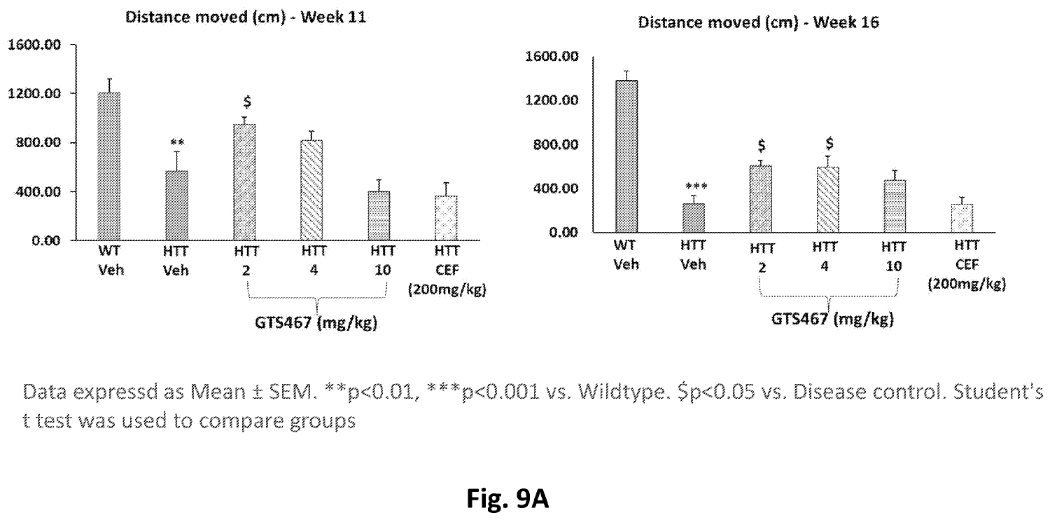

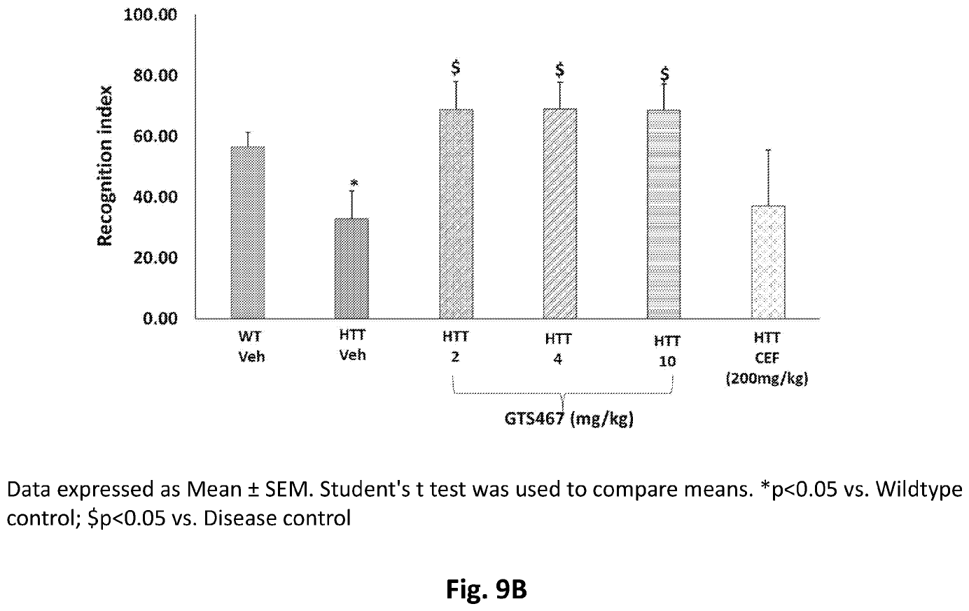

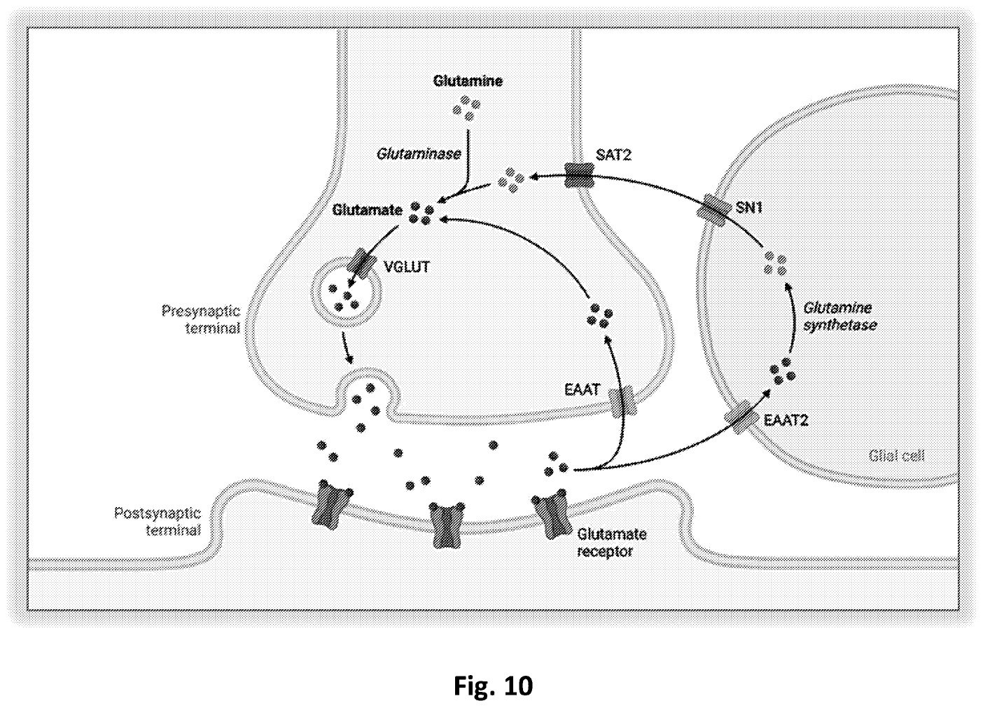

Referring to FIGS. 9A-9B, the present study tested one of the exemplary GLT-1 modulators, GT467, in a mouse model of Huntington's disease.

R6/2 mice model human Huntington's disease by expressing a portion of the human HD gene under human gene promoter elements. Referring to FIGS. 9A-9B, R6/2 mice treated with either vehicle (“HTT Veh”) or 200 mg/kg ceftriaxone (“HTT CEF”) move significantly shorter distances (FIG. 9A) and scored significantly lower in recognition index (FIG. 9B) than the wild-type counterparts treated with vehicle (“WT Veh”). The exemplary GLT-1 modulator GT467, however; significantly increased both the distance (FIG. 9A) and recognition index score (FIG. 9B). These results demonstrate that GT467 significantly improved the locomotor activity and improved the learning and memory impairments in HD mice.

### Example 2: Novel EAAT2 Activator Promotes Glutamate Homeostasis and Improves Cognition Without Producing Impulsive Behaviors in a Rodent Model of Parkinson's Disease

Glutamate induced excitotoxicity has been shown to be one of the causal factors in several neurodegenerative diseases including Parkinson's disease (PD). The excitatory neurotransmitter glutamate is tightly regulated by a network of receptors and transporters in the brain. One such transporter is Excitatory Amino Acid Transporter 2 (EAAT2) which is predominantly localized to astrocytes and is responsible for clearing ˜90% of the glutamate in the synapse. Under conditions of PD, studies have shown that EAAT2 is downregulated, and aberrant activation of presynaptic glutamatergic neurons leads to excitotoxicity and subsequent death of dopaminergic neurons. Dysregulation of dopamine and glutamate neurotransmission are not only implicated in motor and cognitive impairment in PD but also in promoting compulsive and impulsive behaviors. Therefore, the present study hypothesized that small molecule activators of EAAT2 that can effectively reduce excitotoxicity will be beneficial to treat motor and cognitive impairment without promoting impulsive behaviors. The present study tested GTS467—a novel small molecule activator of EAAT2 according to some embodiments in a unilaterally lesioned rodent model of PD. Results from the study confirm that GTS467 significantly improved performance in a 5-choice serial reaction time task with reduced premature impulsive responses and omissions in comparison to vehicle treated PD animals. Ex vivo biochemical analysis of the tissue from prefrontal cortex and striatum from these treated animals showed increase in EAAT2 and a reduction in post synaptic glutamate receptor protein expression with a normalization of signaling functions. These results suggest that GTS467 can be developed as a novel therapeutic to treat excitotoxicity in neurodegenerative diseases.

### Example 2-1

Parkinson's disorder (PD) is a neurodegenerative disorder featuring motor and cognitive impairments with a significant loss of Dopamine (DA) neurons in Substantia nigra. Dopamine agonists are traditionally used in treatment of PD, but they are associated with side effects such as dyskinesia and impulse control disorders (ICDs). Dysregulation of glutamate neurotransmission in PD may be causal for excitotoxicity and ICDs. Studies using NMDA antagonists to treat ICDs was unsuccessful and in some cases exacerbate the effects. Excitatory Amino Acid Transporter 2 (EAAT2) regulates glutamate levels by clearing ˜90% of the glutamate in the synapse. Compound GTS467-a novel small molecule activator of EAAT2 according to some embodiments, was tested. It was hypothesize that activators of EAAT2 will aid in glutamate regulation and reduce excitotoxicity associated with PD.

### Example 2-2: Methods

### Elevated Plus Maze

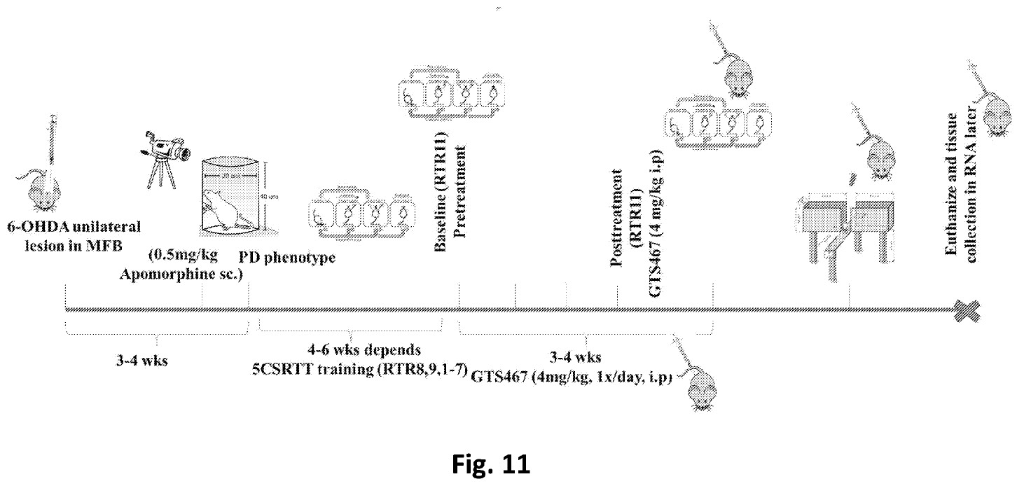

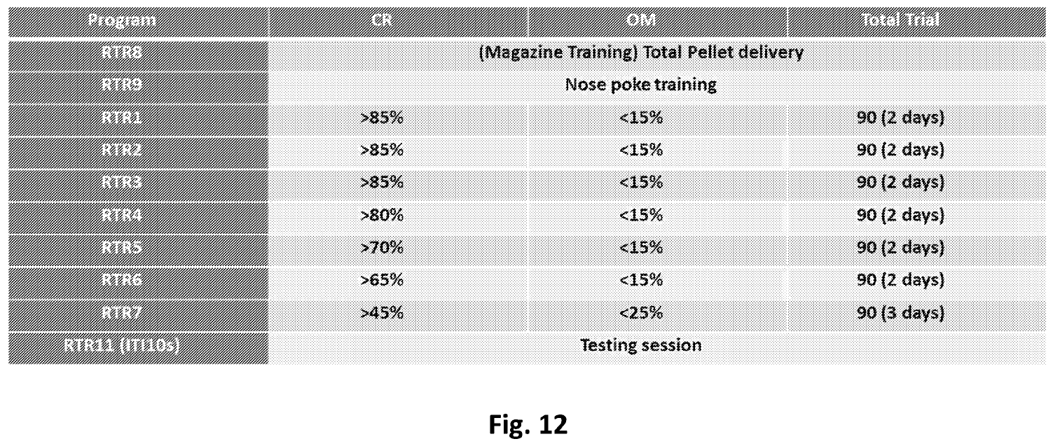

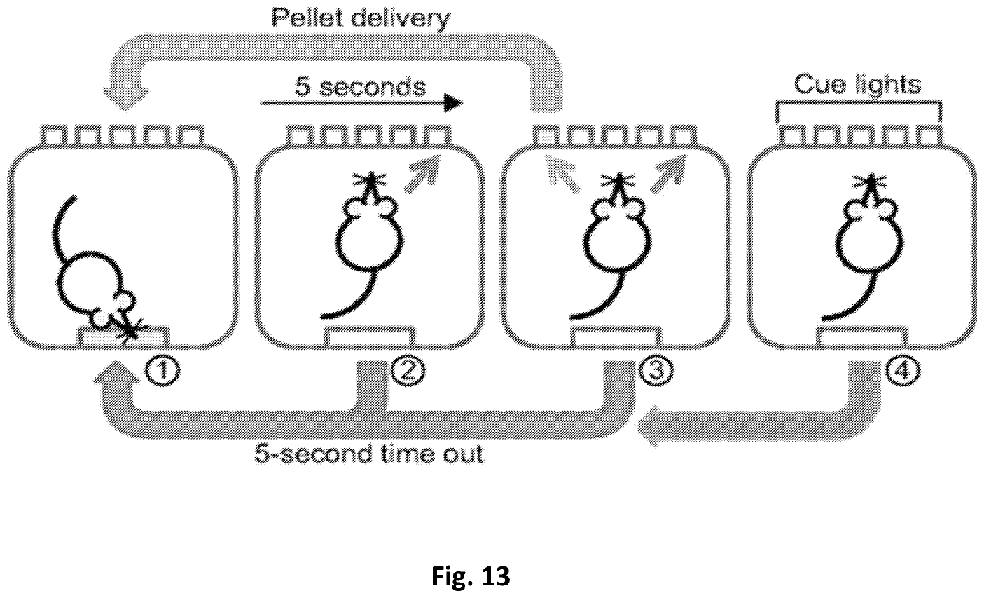

Referring to FIGS. 11-13, in the elevated plus maze test, male PD rats were treated with either saline or GTS467 (4 mg/kg). 10 min post treatment, the rats were placed in the middle of the elevated plus maze (EPM). Time spent in closed and open arms were recorded for 5 min. Frequency of entries in open and close arms was recorded.

Analysis: Animals spending more time in the close arm and less time in the open arm were considered to exhibit anxiety behavior.

### Western Blot Analysis

Cortex and striatum were separately collected, and homogenates were prepared. Homogenates were centrifuged to obtain the protein extract in membrane and cytoplasmic fraction. Protein was estimated for each individual samples. Protein lysates were mixed with 6× loading buffer (5:1 ratio) and were heated (95° C. for 5 min). 20 μg protein was loaded to Tris-Glycine Mini gel for separation at 100 mV for 2 h. Separated proteins were transferred to a PVDF membrane. 3% BSA was used for blocking the nontarget proteins to avoid noise. Membrane was incubated with primary Ab overnight at 4° C. followed by secondary Ab incubation for 3 h at room temperature. The protein bands were detected using ECL methods. The bands were quantified using Image J software.

### Example 2-2: GTS467 Improves Cognitive Function in PD Rats Without Promoting Impulsive Behaviors

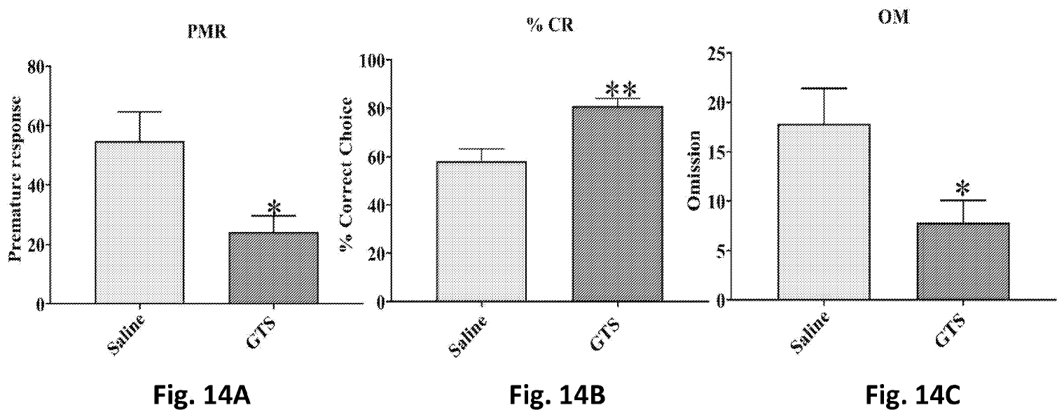

Referring to FIG. 14A, the 5CSRTT assay demonstrate that GTS467 reduces impulsivity (as indicated by the reduced premature responses).

Referring to FIG. 14B, the 5CSRTT assay demonstrate that GTS467 improves cognitive performance (as indicated by the increased percentage of correct choices).

Referring to FIG. 14C, the 5CSRTT assay demonstrate that GTS467 causes a trend towards reducing omissions compared to saline treated PD rats.

### Example 2-3: GTS467 Does not Induce Anxiety in PD Rats

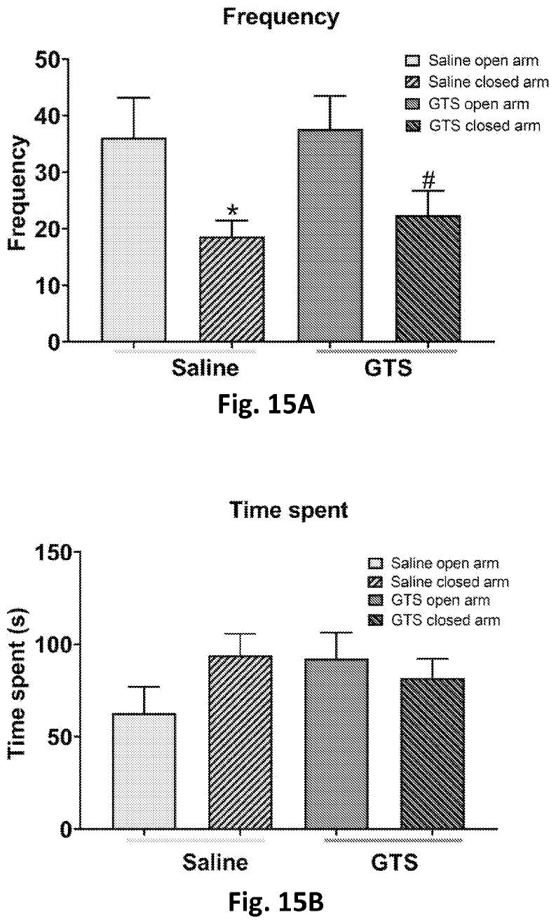

Referring to FIGS. 15A-15B, GTS467 did not induce anxiety in PD rats as estimated in an elevated plus maze (EPM) assay, as no significant difference between the groups in time spent in open vs. closed arm of EPM was observed.

### Example 2-4: GTS467 Treatment Enhances the Expression of EAAT2, Which Results in Normalization of Glutamate Neurotransmission and a Reduction in Impulsive Behaviors

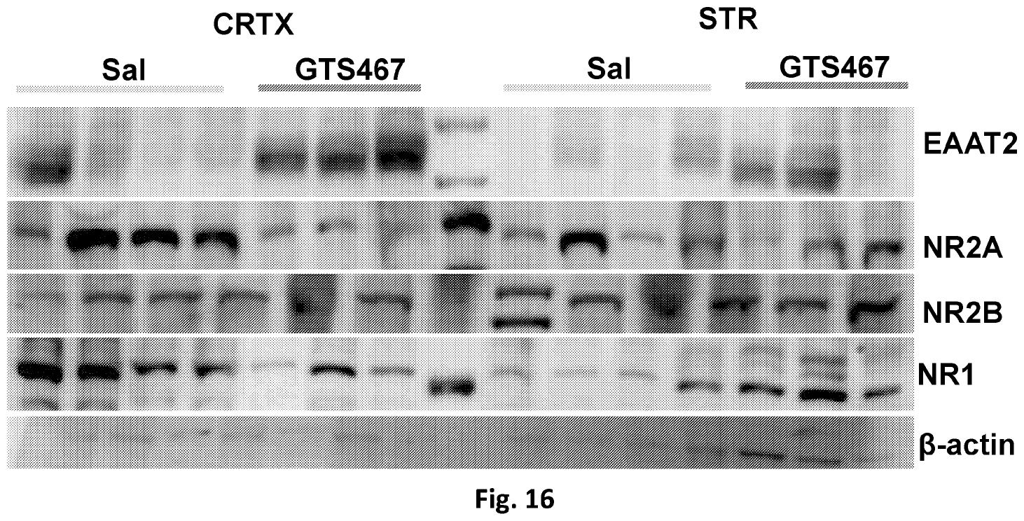

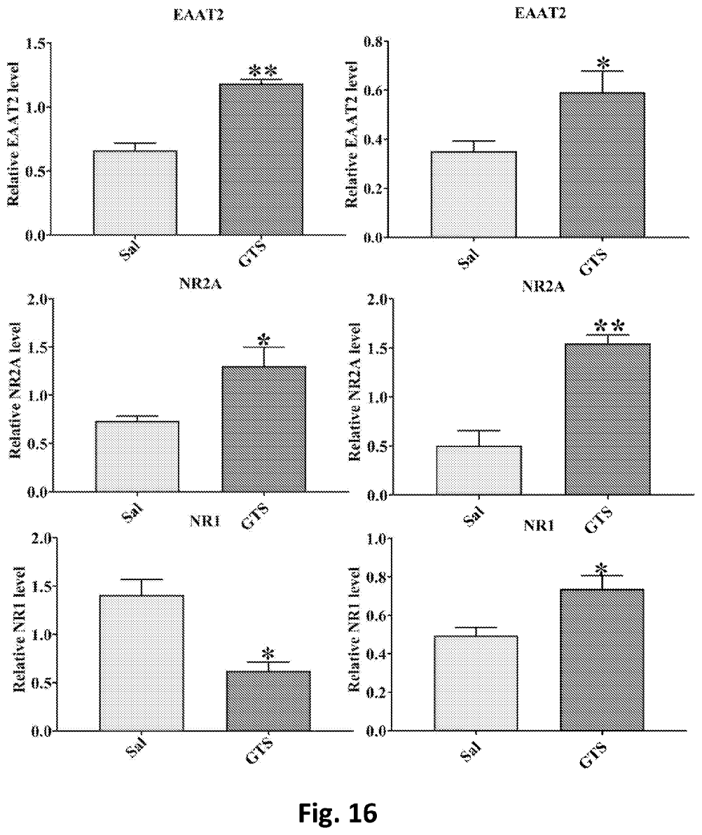

Referring to FIGS. 16-17, GTS467 treatment caused increases in the expression of EAAT2 in both cortex (CRTX) and striatum (STR) in PD rats. GTS467 treatment also caused elevation of NR2A, 2B and NR1 expressions at synaptic level rather at extra synaptic level which promote death of neurons.

Without wishing to be bound by theory, it is hypothesized that the reduction in NR1 may be due to inhibition at projection neuron in prefrontal cortex (PFC) which has been reported to be involved in reducing impulsive action in PD rats.

### ENUMERATED EMBODIMENTS

The following enumerated embodiments are provided, the numbering of which is not to be construed as designating levels of importance:

Embodiment 1: A compound of Formula (I):

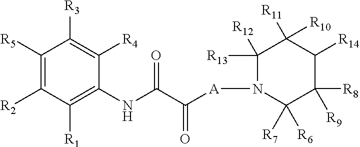

### Formula (I),

or a salt, solvate, enantiomer, diastereoisomer, isotopically labelled derivative, tautomer, or geometric isomer thereof, or mixtures thereof,
wherein:
A is a bond or

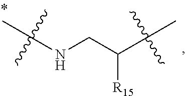

wherein * is the bond to the carbonyl group;
R₁-R₁₃ are each independently selected from the group consisting of H, halogen, OH, C₁-C₆ alkyl, C₁-C₆ alkoxy, C₁-C₆ heteroalkyl, and C₃-C₁₀ cycloalkyl, wherein the alkyl, alkoxy, heteroalkyl, and cycloalkyl are each independently optionally substituted with at least one of C₁-C₆ alkyl, halogen, OH, and C₁-C₆ alkoxy;
R₁₄ is H, C₁-C₆ alkyl, C₁-C₆ heteroalkyl, C₃-C₁₀ cycloalkyl, and phenyl, wherein the alkyl, alkoxy, heteroalkyl, and cycloalkyl are independently optionally substituted with at least one of C₁-C₆ alkyl, halogen, OH, and C₁-C₆ alkoxy;
R₁₅ is phenyl optionally substituted with at least one of C₁-C₆ alkyl, halogen, OH, and C₁-C₆ alkoxy.

Embodiment 2: The compound of Embodiment 1, which prevents, treats and/or ameliorates a neurological disorder.

Embodiment 3: The compound of Embodiment 1, wherein one or more of R₁-R₅ are C₁-C₆ alkoxy.

Embodiment 4: The compound of Embodiment 1, wherein R₁₄ is optionally substituted phenyl.

Embodiment 5: The compound of Embodiment 1, wherein the compound is at least one selected from the group consisting of N1-(4-methoxyphenyl)-N2-(2-(4-methylpiperazin-1-yl)-2-phenylethyl)oxalamide

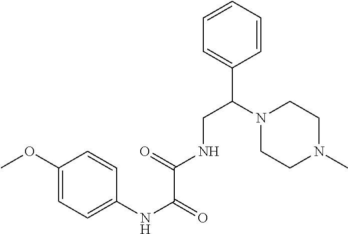

(GT467 or GTS467) and N-(4-methoxyphenyl)-2-oxo-2-(4-phenylpiperazin-1-yl)acetamide

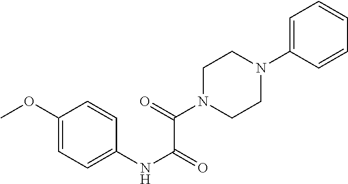 [SMILES: COc1ccc(NC(=O)C(=O)N2CCN(c3ccccc3)CC2)cc1]

(GT511), or a salt, solvate, enantiomer, diastereoisomer, isotopically labelled derivative, tautomer, or geometric isomer thereof, or mixtures thereof.

Embodiment 6: The compound of Embodiment 1, wherein the compound is (R)—N1-(4-methoxyphenyl)-N2-(2-(4-methylpiperazin-1-yl)-2-phenylethyl)oxalamide

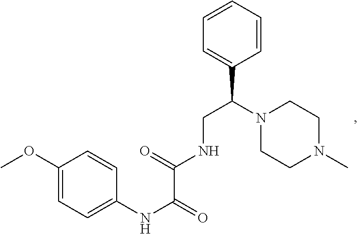

or (S)—N1-(4-methoxyphenyl)-N2-(2-(4-methylpiperazin-1-yl)-2-phenylethyl)oxalamide

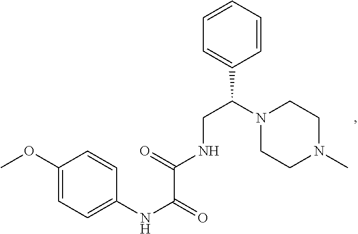

or a salt, solvate, isotopically labelled derivative, or tautomer thereof.

Embodiment 7: A method of increasing activity of GLT-1 protein, the method comprising contacting the GLT-1 protein with the compound of any of Embodiments 1-6.

Embodiment 8: The method of Embodiment 7, wherein the GLT-1 protein is an isolated protein or a protein expressed in a cell.

Embodiment 9: The method of Embodiment 7, wherein the GLT-1 protein is on a surface of an astrocyte.

Embodiment 10: The method of Embodiment 9, wherein the astrocyte is in a central nervous system (CNS) of a subject.

Embodiment 11: The method of Embodiment 10, wherein the subject suffers from a condition associated with excessive extracellular glutamate concentration at a synapse of the CNS, and wherein the method comprises administering to the subject an effective amount of the compound of Embodiment 1.

Embodiment 12: The method of Embodiment 11, wherein the condition associated with excessive extracellular glutamate concentration at the synapse of the CNS comprises at least one neurological disorder selected from the group consisting of Parkinson's disease, Alzheimer's disease, Huntington's disease, amyotrophic lateral sclerosis (ALS), stroke, epilepsy, schizophrenia, compulsive and impulsive drug and alcohol seeking behaviors, and learning and memory impairment associated with neurological and neuropsychiatric disorders.

Embodiment 13: A method of preventing, treating and/or ameliorating neurological disorder in a subject in need thereof, the method comprises administering to the subject an effective amount of the compound of any of Embodiments 1-6.

Embodiment 14: The method of Embodiment 13, wherein the neurological disorder comprises at least one selected from the group consisting of Parkinson's disease, Alzheimer's disease, Huntington's disease, amyotrophic lateral sclerosis (ALS), stroke, epilepsy, schizophrenia, compulsive and impulsive drug and alcohol seeking behaviors, and/or learning and memory impairment associated with neurological and neuropsychiatric disorders.

Embodiment 15: The method of Embodiment 13, wherein the compound is administered to the subject as a pharmaceutical composition.

Embodiment 16: The method of Embodiment 15, wherein the pharmaceutical composition further comprises a pharmaceutically acceptable carrier.

Embodiment 17: The method of Embodiment 13, wherein the method further comprises administering to the subject an additional therapeutic agent.

Embodiment 18: The method of Embodiment 17, wherein the compound and the additional therapeutic agent are co-administered to the subject.

Embodiment 19: The method of Embodiment 18, wherein the compound and the additional therapeutic agent are co-formulated.

Embodiment 20: The method of Embodiment 13, wherein the subject is a human.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

1. A compound of Formula (I): 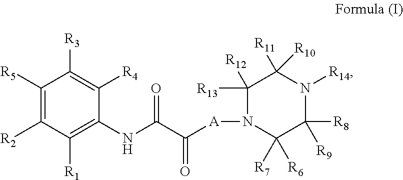
or a pharmaceutically acceptable salt, solvate, enantiomer, diastereoisomer, isotopically labelled derivative, or tautomer thereof,
wherein:
A is a bond or 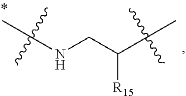
wherein the bond marked with * is connected to the carbonyl group;
R₁-R₁₃ are each independently selected from the group consisting of H, halogen, OH, C₁-C₆ alkyl, C₁-C₆ alkoxy, C₁-C₆ heteroalkyl, and C₃-C₁₀ cycloalkyl, wherein the alkyl, alkoxy, heteroalkyl, and cycloalkyl are each independently optionally substituted with at least one of C₁-C₆ alkyl, halogen, OH, and C₁-C₆ alkoxy;
R₁₄ is H, C₁-C₆ alkyl, C₁-C₆ heteroalkyl, C₃-C₁₀ cycloalkyl, and phenyl, wherein the alkyl, alkoxy, heteroalkyl, and cycloalkyl are independently optionally substituted with at least one of C₁-C₆ alkyl, halogen, OH, and C₁-C₆ alkoxy; and
R₁₅ is phenyl optionally substituted with at least one of C₁-C₆ alkyl, halogen, OH, and C₁-C₆ alkoxy.

2. (canceled)

3. The compound of claim 1, wherein one or more of R₁-R₅ are C₁-C₆ alkoxy.

4. The compound of claim 1, wherein R₁₄ is optionally substituted phenyl.

5. The compound of claim 1, wherein the compound is at least one selected from the group consisting of
N1-(4-methoxyphenyl)-N2-(2-(4-methylpiperazin-1-yl)-2-phenylethyl)oxalamide 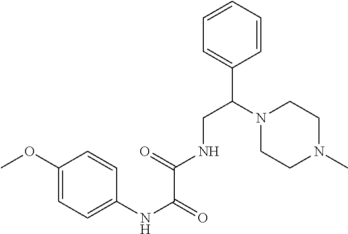
(GT467 or GTS467) and
N-(4-methoxyphenyl)-2-oxo-2-(4-phenylpiperazin-1-yl)acetamide 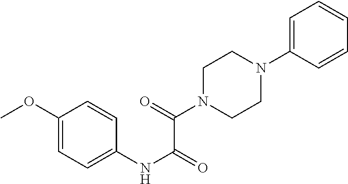 [SMILES: COc1ccc(NC(=O)C(=O)N2CCN(c3ccccc3)CC2)cc1]
(GT511),
or a pharmaceutically acceptable salt, solvate, enantiomer, diastereoisomer, isotopically labelled derivative, or tautomer thereof.

6. The compound of claim 1, wherein the compound is (R)—N1-(4-methoxyphenyl)-N2-(2-(4-methylpiperazin-1-yl)-2-phenylethyl)oxalamide 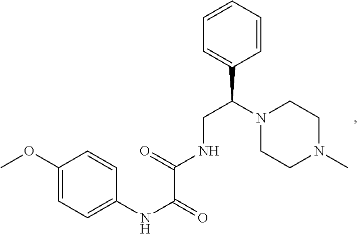
or
(S)—N1-(4-methoxyphenyl)-N2-(2-(4-methylpiperazin-1-yl)-2-phenylethyl)oxalamide 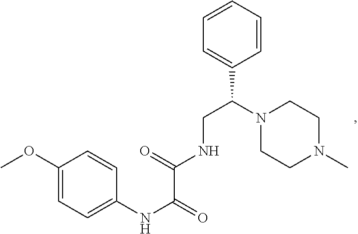
or a pharmaceutically acceptable salt, solvate, isotopically labelled derivative, or tautomer thereof.

7. A method of increasing activity of a GLT-1 protein, the method comprising:
contacting the GLT-1 protein with a compound of Formula (I): 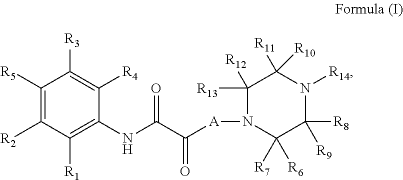
or a pharmaceutically acceptable salt, solvate, enantiomer, diastereoisomer, isotopically labelled derivative, or tautomer thereof,
wherein:
A is a bond or 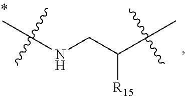
wherein the bond marked with * is connected to a carbonyl group;
R₁-R₁₃ are each independently selected from the group consisting of H, halogen, OH, C₁-C₆ alkyl, C₁-C₆ alkoxy, C₁-C₆ heteroalkyl, and C₃-C₁₀ cycloalkyl, wherein the alkyl, alkoxy, heteroalkyl, and cycloalkyl are each independently optionally substituted with at least one of C₁-C₆ alkyl, halogen, OH, and C₁-C₆ alkoxy;
R₁₄ is H, C₁-C₆ alkyl, C₁-C₆ heteroalkyl, C₃-C₁₀ cycloalkyl, and phenyl, wherein the alkyl, alkoxy, heteroalkyl, and cycloalkyl are independently optionally substituted with at least one of C₁-C₆ alkyl, halogen, OH, and C₁-C₆ alkoxy; and
R₁₅ is phenyl optionally substituted with at least one of C₁-C₆ alkyl, halogen, OH, and C₁-C₆ alkoxy.

8. The method of claim 7, wherein the GLT-1 protein is an isolated protein or a protein expressed in a cell.

9. The method of claim 7, wherein the GLT-1 protein is on a surface of an astrocyte.

10. The method of claim 9, wherein the astrocyte is in a central nervous system (CNS) of a subject.

11. The method of claim 10, wherein the subject suffers from a condition associated with excessive extracellular glutamate concentration at a synapse of the CNS, and wherein the method comprises administering to the subject an effective amount of the compound of claim 1.

12. The method of claim 11, wherein the condition associated with excessive extracellular glutamate concentration at the synapse of the CNS is at least one neurological disorder selected from the group consisting of Parkinson's disease, Alzheimer's disease, Huntington's disease, amyotrophic lateral sclerosis (ALS), stroke, epilepsy, schizophrenia, compulsive and impulsive drug and alcohol seeking behaviors, and learning and memory impairment associated with neurological and neuropsychiatric disorders.

13. A method of treating or ameliorating neurological disorder in a subject in need thereof, the method comprising:
administering to the subject a therapeutically effective amount of a compound of Formula (I): 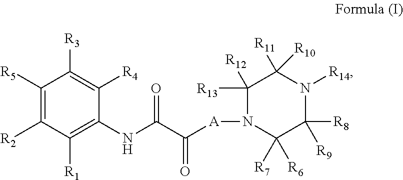
or a pharmaceutically acceptable salt, solvate, enantiomer, diastereoisomer, isotopically labelled derivative, or tautomer thereof.
wherein:
A is a bond or 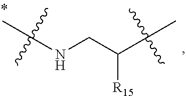
wherein the bond marked with * is connected to a carbonyl group;
R₁-R₁₃ are each independently selected from the group consisting of H, halogen, OH, C₁-C₆ alkyl, C₁-C₆ alkoxy, C₁-C₆ heteroalkyl, and C₃-C₁₀ cycloalkyl, wherein the alkyl, alkoxy, heteroalkyl, and cycloalkyl are each independently optionally substituted with at least one of C₁-C₆ alkyl, halogen, OH, and C₁-C₆ alkoxy;
R₁₄ is H, C₁-C₆ alkyl, C₁-C₆ heteroalkyl, C₃-C₁₀ cycloalkyl, and phenyl, wherein the alkyl, alkoxy, heteroalkyl, and cycloalkyl are independently optionally substituted with at least one of C₁-C₆ alkyl, halogen, OH, and C₁-C₆ alkoxy; and
R₁₅ is phenyl optionally substituted with at least one of C₁-C₆ alkyl, halogen, OH, and C₁-C₆ alkoxy.

14. The method of claim 13, wherein the neurological disorder is at least one selected from the group consisting of Parkinson's disease, Alzheimer's disease, Huntington's disease, amyotrophic lateral sclerosis (ALS), stroke, epilepsy, schizophrenia, compulsive and impulsive drug and alcohol seeking behaviors, and learning and memory impairment associated with neurological and neuropsychiatric disorders.

15. The method of claim 13, wherein the compound is administered to the subject as a pharmaceutical composition.

16. The method of claim 15, wherein the pharmaceutical composition further comprises at least one pharmaceutically acceptable carrier.

17. The method of claim 13, wherein the method further comprises administering to the subject an additional therapeutic agent.

18. The method of claim 17, wherein the compound and the additional therapeutic agent are co-administered to the subject.

19. The method of claim 18, wherein the compound and the additional therapeutic agent are co-formulated.

20. The method of claim 13, wherein the subject is a human.
